(19) United States
(12) Patent Application Publication
(10) Pub. No.: US 2025/0361567 A1
(43) Pub. Date: Nov. 27, 2025

(54) METHODS FOR DETECTING MUTATIONAL SIGNATURES USING TARGETED PANELS

(71) Applicant: Life Technologies Corporation, Carlsbad, CA (US)

(72) Inventors: Fiona Hyland, San Mateo, CA (US); Ajithavalli Chellappan, Bengaluru (IN); Chintan Vora, Fremont, CA (US); Shilpa Nair, Delhi (IN); Jagannath Patro, Bengaluru (IN); Ritika Raj, Bengaluru (IN); Rushikesh Kanap, Bengaluru (IN)

(21) Appl. No.: 19/227,705

(22) Filed: Jun. 4, 2025

Related U.S. Application Data
(63) Continuation of application No. 17/307,152, filed on May 4, 2021.

(51) Int. Cl. C12Q 1/6886

(52) U.S. Cl. CPC C12Q 1/6886; C12Q 2600/156

(57) ABSTRACT

A targeted panel with low sample input requirements from a tumor sample may be processed to identify the presence of a mutational signature. The method may include the steps of: amplifying nucleic acid sequences at targeted locations in the tumor sample genome by a targeted panel to generate nucleic acid sequence reads, detecting variants in the nucleic acid sequence reads, generating a set of trinucleotides by appending flanking 5′ and 3′ bases to each variant, determining a frequency of each trinucleotide to form a mutation matrix, determining a cosine similarity value of the mutation matrix and each mutational signature in a matrix of mutational signatures to form a matrix of similarity values, and selecting mutational signatures from the matrix of mutational signatures when a corresponding cosine similarity value is greater than or equal to a threshold to indicate presence of the selected mutational signatures in the tumor sample genome.

### FIELD

This application generally relates to methods, systems, and computer-readable media for detection of mutational signatures, and, more specifically, to methods, systems, and computer-readable media for detection of mutational signatures based on nucleic acid sequencing data obtained using targeted panels and next-generation sequencing technology or systems.

### SUMMARY

Mutational signatures are mutation profiles identifiable based on specific causes of somatic mutations in tumor cells and driven by mutational processes. These mutational processes may be environmental in origin (UV damage, tobacco smoking damage, environmental mutagens) or biological (defects in mismatch repair genes). The presence of a mutational signature in a sample can provide information useful for understanding the biological process behind cancer mutagenesis and driver mutation origin. Mutational signatures are generally determined from Whole Genome Sequencing (WGS) or Whole Exome Sequencing (WES) data. Systems and methods described herein are applied to amplification-based targeted sequencing data to predict mutational signatures, instead of using WES or WGS. Systems and methods using amplification-based targeted sequencing data to predict mutational signatures, rather than WGS or WES, are advantageous because of the limited availability of DNA in formalin-fixed paraffin-embedded (FFPE) samples and the higher success rates of targeted amplicon-based sequencing. There is a need for new and improved methods, systems, and computer-readable media for detection of mutational signatures using targeted panels to generate targeted sequencing data from the tumor sample genome.

According to an exemplary embodiment, there is provided a method of analyzing a tumor sample genome for a mutational signature, including the following steps: (1) amplifying nucleic acid sequences at targeted locations in the tumor sample genome by a targeted panel to generate a plurality of nucleic acid sequence reads; (2) detecting variants in the plurality of nucleic acid sequence reads to produce a plurality of variants; (3) generating a set of trinucleotides by appending a flanking 5′ base and a flanking 3′base to each variant: (4) determining a frequency of each trinucleotide in the set of trinucleotides to form a mutation matrix; (5) determining a cosine similarity value of the mutation matrix and each mutational signature in a matrix of mutational signatures to form a matrix of similarity values; and (6) selecting one or more mutational signatures from the matrix of mutational signatures when a corresponding cosine similarity value in the matrix of similarity values is greater than or equal to a threshold to indicate a presence of one or more selected mutational signatures in the tumor sample genome.

According to an exemplary embodiment, there is provided a system for analyzing a tumor sample genome for a mutational signature, comprising a processor and a data store communicatively connected with the processor, the processor configured to execute instructions, which, when executed by the processor, cause the system to perform a method, including: (1) amplifying nucleic acid sequences at targeted locations in the tumor sample genome by a targeted panel to generate a plurality of nucleic acid sequence reads; (2) detecting variants in the plurality of nucleic acid sequence reads to produce a plurality of variants; (3) generating a set of trinucleotides by appending a flanking 5′ base and a flanking 3′ base to each variant; (4) determining a frequency of each trinucleotide in the set of trinucleotides to form a mutation matrix; (5) determining a cosine similarity value of the mutation matrix and each mutational signature in a matrix of mutational signatures to form a matrix of similarity values; and (6) selecting one or more mutational signatures from the matrix of mutational signatures when a corresponding cosine similarity value in the matrix of similarity values is greater than or equal to a threshold to indicate a presence of one or more selected mutational signatures in the tumor sample genome.

According to an exemplary embodiment, there is provided a non-transitory machine-readable storage medium comprising instructions which, when executed by a processor, cause the processor to perform a method for analyzing a tumor sample genome for a mutation load, including: (1) receiving a plurality of nucleic acid sequence reads, wherein the nucleic acid sequence reads correspond to a plurality of targeted locations in the tumor sample genome (2) detecting variants in the plurality of nucleic acid sequence reads to produce a plurality of variants; (3) generating a set of trinucleotides by appending a flanking 5′ base and a flanking 3′ base to each variant: (4) determining a frequency of each trinucleotide in the set of trinucleotides to form a mutation matrix; (5) determining a cosine similarity value of the mutation matrix and each mutational signature in a matrix of mutational signatures to form a matrix of similarity values; and (6) selecting one or more mutational signatures from the matrix of mutational signatures when a corresponding cosine similarity value in the matrix of similarity values is greater than or equal to a threshold to indicate a presence of one or more selected mutational signatures in the tumor sample genome.

### BRIEF DESCRIPTION OF THE DRAWINGS

The novel features are set forth with particularity in the appended claims. A better understanding of the features and advantages will be obtained by reference to the following detailed description that sets forth illustrative embodiments, in which the principles of the invention are utilized, and the accompanying drawings of which:

FIG. 1 is a block diagram showing a method for detecting and filtering variants from aligned sequence reads from the targeted panel, according to an exemplary embodiment.

FIG. 2 is a block diagram of a method for detecting mutational signatures from the variant list, in accordance with an embodiment.

FIG. 3A gives an example of a plot of the ratios of the frequencies of the trinucleotides in an hg19 reference genome to the frequencies of the trinucleotides in the portion of the genome covered by the TML panel.

FIG. 3B gives an example of a plot of the ratios of the frequencies of the trinucleotides in an hg19reference genome to the frequencies of the trinucleotides in the portion of the genome covered by the OCAPlus panel.

FIG. 4 gives examples of heat maps of the cosine similarity values calculated from sequencing data for a whole genome, the TML panel with normalization and the TML panel without normalization.

FIG. 5A shows an example of a pie chart showing the contributions of the selected COSMIC mutational signatures using for the TML targeted panel without the normalizing step.

FIG. 5B is an example of a plot of the trinucleotide frequencies for the TML targeted panel without the normalizing step.

FIG. 6A shows an example of a pie chart showing the contributions of the selected COSMIC mutational signatures using for the TML targeted panel with the normalizing step.

FIG. 6B is an example of a plot of the trinucleotide frequencies for the TML targeted panel with the normalizing step.

FIG. 7A shows an example of a pie chart showing the contributions of the selected COSMIC mutational signatures using whole genome sequencing data.

FIG. 7B is an example of a plot of the trinucleotide frequencies for the whole genome sequencing data.

FIGS. 8A, 8B, 8C, 8D-1, 8D-2, 8E-1, 8E-2, 8F-1 and 8F-2 show examples of results that may be included in a display for the user.

FIGS. 9A-1, 9A-2, 9B-1, 9B-2, 9C and 9D show results for a sample with high cosine similarity =0.8046 for MMR signature SBS14 and an MMR gene MSH2 mutation.

FIGS. 10A-1, 10A-2, 10B-1, 10B-2, 10C and 10D show results for a sample with high cosine similarity=0.7737 for MMR signature SBS44 and an MMR gene MSH2 mutation.

FIGS. 11A-1, 11A-2, 11B-1, 11B-2, 11C and 11D show results for a sample with cosine similarity=0.7691 for SBS36 signature and MUTYH mutations.

FIGS. 12A-1, 12A-2, 12B-1, 12B-2, 12C and 12D show results for a sample with cosine similarity=0.7634 for SBS5 signature and expected ERCC2 gene mutations.

FIGS. 13A-1, 13A-2, 13B-1, 13B-2, 13C, 13D-1, 13D-2, 13E and 13F show results for two samples for SBS30 base excision repair signature and expected NTHL1 mutations.

FIG. 14 is a table of results showing the prevalence of base excision repair, MMR, APOBEC and HRR signatures in data obtained from samples sequenced using the OCAPlus panel and data obtained from samples sequenced using the TML panel.

FIG. 15 gives results of mutational signature predictions applied to two tumor samples and a control.

FIG. 16 shows an example of results that compare detections of mutational signatures in tumor samples and matched normal samples.

FIG. 17 is a schematic diagram of an exemplary system for reconstructing a nucleic acid sequence, in accordance with various embodiments.

FIG. 18 is a schematic diagram of a system for annotating genomic variants, in accordance with various embodiments.

### DETAILED DESCRIPTION

In accordance with the teachings and principles embodied in this application, new methods, systems and non-transitory machine-readable storage medium are provided to detect mutational signatures by analysis of variants in nucleic acid sequence reads generated from a sample using a targeted panel.

In various embodiments, DNA (deoxyribonucleic acid) may be referred to as a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. In various embodiments, “nucleic acid sequencing data,” “nucleic acid sequencing information,” “nucleic acid sequence,” “genomic sequence,” “genetic sequence,” or “fragment sequence,” “nucleic acid sequence read” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

The phrase “base space” refers to a nucleic acid sequence data schema where nucleic acid sequence information is represented by the actual nucleotide base composition of the nucleic acid sequence. For example, the nucleic acid sequence “ATCGA” is represented in base space by the actual nucleotide base identities (for example, A, T/or U, C, G) of the nucleic acid sequence.

The phrase “flow space” refers to a nucleic acid sequence data schema wherein nucleic acid sequence information is represented by nucleotide base identifications (or identifications of known nucleotide base flows) coupled with signal or numerical quantification components representative of nucleotide incorporation events for the nucleic acid sequence. The quantification components may be related to the relative number of continuous base repeats, such as homopolymers, whose incorporation is associated with a respective nucleotide base flow. For example, the nucleic acid sequence “ATTTGA” may be represented by the nucleotide base identifications A, T, G and A (based on the nucleotide base flow order) plus a quantification component for the various flows indicating base presence/absence as well as possible existence of homopolymers. Thus for “T” in the example sequence above, the quantification component may correspond to a signal or numerical identifier of greater magnitude than would be expected for a single “T” and may be resolved to indicate the presence of a homopolymer stretch of “T”s (in this case a 3-mer) in the “ATTTGA” nucleic acid sequence.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, for example 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine. “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The phrase “genomic variants” or “genome variants” denote a single or a grouping of sequences (in DNA or RNA) that have undergone changes as referenced against a particular species or sub-populations within a particular species due to mutations, recombination/crossover or genetic drift. Examples of types of genomic variants include, but are not limited to single nucleotide polymorphisms (SNPs), copy number variations (CNVs), insertions/deletions (indels), single nucleotide variant (SNVs), multiple nucleotide variants (MNVs), inversions, etc.

The abbreviation “APOBEC” is for “apolipoprotein B mRNA editing enzyme, catalytic polypeptide-like”. The abbreviation “HRR” is for “homologous recombinational repair”. The abbreviation MMR is for “mismatch repair”.

In various embodiments, genomic variants can be detected using a nucleic acid sequencing system and/or analysis of sequencing data. The sequencing workflow can begin with the test sample being sheared or digested into hundreds, thousands or millions of smaller fragments which are sequenced on a nucleic acid sequencer to provide hundreds, thousands or millions of sequence reads, such as nucleic acid sequence reads. Each read can then be mapped to a reference or target genome, and in the case of mate-pair fragments, the reads can be paired thereby allowing interrogation of repetitive regions of the genome. The results of mapping and pairing can be used as input for various standalone or integrated genome variant (for example, SNP, CNV, Indel, inversion, etc.) analysis tools.

The phrase “sample genome” can denote a whole or partial genome of an organism.

The term “allele” as used herein refers to a genetic variation associated with a gene or a segment of DNA, i.e., one of two or more alternate forms of a DNA sequence occupying the same locus.

The term “locus” as used herein refers to a specific position on a chromosome or a nucleic acid molecule. Alleles of a locus are located at identical sites on homologous chromosomes.

As used herein, a “targeted panel” refers to a set of target-specific primers that are designed for selective amplification of target gene sequences in a sample. In some embodiments, following selective amplification of at least one target sequence, the workflow further includes nucleic acid sequencing of the amplified target sequence.

As used herein, “target sequence” or “target gene sequence” and its derivatives, refers to any single or double-stranded nucleic acid sequence that can be amplified or synthesized according to the disclosure, including any nucleic acid sequence suspected or expected to be present in a sample. In some embodiments, the target sequence is present in double-stranded form and includes at least a portion of the particular nucleotide sequence to be amplified or synthesized, or its complement, prior to the addition of target-specific primers or appended adapters. Target sequences can include the nucleic acids to which primers useful in the amplification or synthesis reaction can hybridize prior to extension by a polymerase. In some embodiments, the term refers to a nucleic acid sequence whose sequence identity, ordering or location of nucleotides is determined by one or more of the methods of the disclosure.

As used herein, “target-specific primer” and its derivatives, refers to a single stranded or double-stranded polynucleotide, typically an oligonucleotide, that includes at least one sequence that is at least 50% complementary, typically at least 75% complementary or at least 85% complementary, more typically at least 90% complementary, more typically at least 95% complementary, more typically at least 98% or at least 99% complementary, or identical, to at least a portion of a nucleic acid molecule that includes a target sequence. In such instances, the target-specific primer and target sequence are described as “corresponding” to each other. In some embodiments, the target-specific primer is capable of hybridizing to at least a portion of its corresponding target sequence (or to a complement of the target sequence); such hybridization can optionally be performed under standard hybridization conditions or under stringent hybridization conditions. In some embodiments, the target-specific primer is not capable of hybridizing to the target sequence, or to its complement, but is capable of hybridizing to a portion of a nucleic acid strand including the target sequence, or to its complement. In some embodiments, a forward target-specific primer and a reverse target-specific primer define a target-specific primer pair that can be used to amplify the target sequence via template-dependent primer extension. Typically, each primer of a target-specific primer pair includes at least one sequence that is substantially complementary to at least a portion of a nucleic acid molecule including a corresponding target sequence but that is less than 50% complementary to at least one other target sequence in the sample. In some embodiments, amplification can be performed using multiple target-specific primer pairs in a single amplification reaction, wherein each primer pair includes a forward target-specific primer and a reverse target-specific primer, each including at least one sequence that substantially complementary or substantially identical to a corresponding target sequence in the sample, and each primer pair having a different corresponding target sequence. In various embodiments, target nucleic acids generated by the amplification of multiple target-specific sequences from a population of nucleic acid molecules can be sequenced. In some embodiments, the amplification can include hybridizing one or more target-specific primer pairs to the target sequence, extending a first primer of the primer pair, denaturing the extended first primer product from the population of nucleic acid molecules, hybridizing to the extended first primer product the second primer of the primer pair, extending the second primer to form a double stranded product, and digesting the target-specific primer pair away from the double stranded product to generate a plurality of amplified target sequences. In some embodiments, the amplified target sequences can be ligated to one or more adapters. In some embodiments, the adapters can include one or more nucleotide barcodes or tagging sequences. In some embodiments, the amplified target sequences once ligated to an adapter can undergo a nick translation reaction and/or further amplification to generate a library of adapter-ligated amplified target sequences. Exemplary methods of multiplex amplification are described in U.S. application Ser. No. 13/458,739 filed Nov. 12, 2012 and titled “Methods and Compositions for Multiplex PCR”,

In various embodiments, the method of performing multiplex PCR amplification includes contacting a plurality of target-specific primer pairs having a forward and reverse primer, with a population of target sequences to form a plurality of template/primer duplexes; adding a DNA polymerase and a mixture of dNTPs to the plurality of template/primer duplexes for sufficient time and at sufficient temperature to extend either (or both) the forward or reverse primer in each target-specific primer pair via template-dependent synthesis thereby generating a plurality of extended primer product/template duplexes; denaturing the extended primer product/template duplexes; annealing to the extended primer product the complementary primer from the target-specific primer pair; and extending the annealed primer in the presence of a DNA polymerase and dNTPs to form a plurality of target-specific double-stranded nucleic acid molecules.

Systems and methods described herein are applied to amplification-based targeted sequencing data to predict mutational signatures, instead of using WES or WGS. The input DNA required for WES or WGS is approximately 50-100 ng. Amplification-based targeted sequencing data may be produced by a targeted panel using as little as 20 ng of DNA. For example, a targeted panel such as Oncomine Tumor Mutation Load Assay™ (TML) (Thermo Fisher Scientific, Cat. Nos. A37909 and A37910), a targeted next-generation sequencing (NGS) assay covering 1.65 megabases (Mb) across 409 oncogenes, may be used to provide targeted sequencing data from a tumor sample, with as little as 20 ng of input DNA, for predicting mutational signatures. For example, a targeted panel such as the Oncomine Comprehensive Assay Plus™ (Thermo Fisher Scientific, Cat. Nos. A49667, A49671, A48578 and A48577) is a targeted next-generation sequencing (NGS) assay that may be used to provide targeted sequencing data from a tumor sample, with as little as 20 ng of input DNA, for predicting mutational signatures. The Oncomine Comprehensive Assay Plus™ (OCAPlus) provides a comprehensive genomic profiling solution appropriate for FFPE tissues. The assay addresses multiple biomarkers covering over 500 genes, including targets that are relevant in cancer. This assay enables analysis of variants across 500+ genes and detection of SNVs, CNVs, In-Dels, TMB, MSI, and gene fusions. In some embodiments, the panel may comprise a custom panel or other targeted panel of cancer driver genes or other genes associated with cancer.

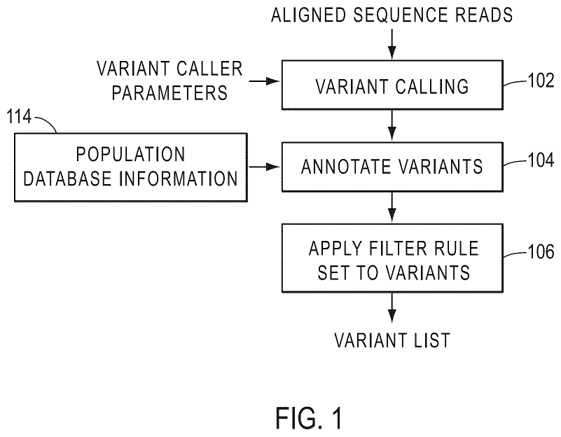

FIG. 1 is a block diagram of processing steps for detecting and filtering variants from aligned sequence reads from the targeted panel, according to an exemplary embodiment. In the variant calling step 102, a processor receives aligned sequence reads resulting from alignment of sequence reads from targeted sequencing of a tumor sample. The aligned sequence reads can be retrieved from a file using a BAM file format, for example. The aligned sequence reads may correspond to a plurality of targeted locations in the tumor sample genome. The variant calling step 102 may be configured by one or more variant caller parameters. In some embodiments, variant caller parameters may include parameters for minimum allele frequency, minimum read depth and data quality stringency. The minimum allele frequency parameter sets the minimum observed allele frequency required for a non-reference variant call. The data quality stringency parameter sets a threshold for read quality required to make a variant call. In some embodiments, the variant caller parameters may be set to the exemplary values given in Table 1.

TABLE 1

| | Parameter Value | | | |
|---|---|---|---|---|
| Variant Caller Parameter | SNV | Indel | Hotspot | Range |
| Minimum Allele Frequency | 0.035 | 0.1 | 0.03 | 0.001 to 0.15 |
| Minimum Read Depth | 40 | 15 | 15 | 10-60 |
| Data Quality Stringency | 11 | 11 | 11 | 5 to 25 |

In some embodiments, variant caller parameters may include a minimum coverage parameter, or minimum read depth parameter, that sets a minimum coverage required for a variant to be called. The minimum coverage parameter may be set to levels to reduce C>T or G>A type nonsystematic noise. The minimum coverage parameter may be set in a range from 10 to 60. The minimum coverage parameter of 20 gives a 10% level of detection (LOD) and minimum coverage parameter of 60 gives a 5% level of LOD.

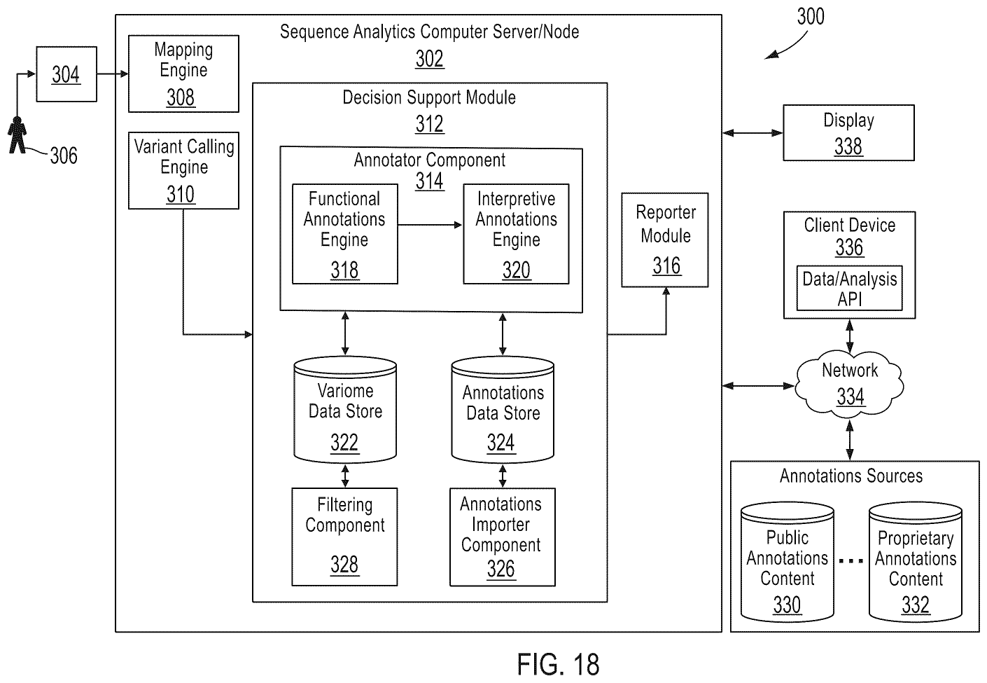

In some embodiments the aligned sequence reads are provided by the mapping engine 308 described with respect to FIG. 18. In some embodiments the variant calling step 102 may be implemented by the variant calling engine 310 described with respect to FIG. 18. In some embodiments, the variant detection methods for use with the present teachings may include one or more features described in U.S. Pat. Appl. Publ. No. 2013/0345066, published Dec. 26, 2013, U.S. Pat. Appl. Publ. No. 2014/0296080, published Oct. 2, 2014, and U.S. Pat. Appl. Publ. No. 2014/0052381, published Feb. 20, 2014, each of which incorporated by reference herein in its entirety. In some embodiments, other variant detection methods may be used. In various embodiments, a variant caller can be configured to communicate variants called for a sample genome as a *.vcf, *gff, or *.hdf data file. The called variant information can be communicated using any file format as long as the called variant information can be parsed and/or extracted for analysis.

Returning to FIG. 1, in the variant annotating step 104, the processor annotates the detected variants with information associated with the respective variants from one or more population databases. In some embodiments, the annotation information may include the minor allele frequency (MAF) of the variant. The population database may provide public annotation information content or proprietary annotation information content. For example, publicly available population databases include: 5000exomes—NHLBI Exome Sequencing Project (http://evs.gs.washington.edu/EVS/), 1000 genomes—International Genome Sample Resource (IGSR) (http://www.internationalgenome.org/home) and ExAC—Exome Aggregation Consortium (http://exac.broadinstitute.org) and UCSC common SNPs (www.genome.ucsc.edu/). Annotation information from other population databases in addition to or in place of these databases may be used. It may be understood that as genetic information resources develop new and more extensive databases may become available.

In some embodiments the annotating step 104 may be implemented in the annotator component 314 and the population database information may be stored in annotations data store 324 described with respect to FIG. 18. In some embodiments, the annotation methods for use with the present teachings may include one or more features described in U.S. Pat. Appl. Publ. No. 2016/0026753, published Jan. 28, 2016, incorporated by reference herein in its entirety.

In the filtering step 106, the processor applies a rule set to retain somatic variants and remove germline variants from the detected variants. In some embodiments, a filter rule set is applied to each detected variant and includes at least some of the rules listed in Table 2.

TABLE 2

| | Filter Rule |
|---|---|
| 1. | Retain one or more variant types selected from SNVs, indels |
| | and MNVs; optionally filter out other variant types |
| 2. | Filter out SNVs inside homopolymers with lengths greater |
| | than 7. |
| 3. | Retain variants found in 1000 genomes with MAF in a given |
| | MAF range; filter out variants outside the MAF range. |
| 4. | Retain variants found in 5000 exomes with MAF in a given |
| | MAF range; filter out variants outside the MAF range. |
| 5. | Retain variants found in ExAC with MAF in a given MAF |
| | range; filter out variants outside the MAF range. |
| 6. | Filter out variants found in UCSC common SNPs. |

In some embodiments, particular variant types are retained, such as SNVs only, SNVs and indels, or SNVs, indels and MNVs, for further analysis while other types of variants are filtered out. In some embodiments, variants in regions with homopolymer lengths greater than 7 are filtered out to mitigate lower accuracy in base calling for long homopolymers. In filter rules 3, 4 and 5, detected variants are retained if the MAF indicated by the population database is within a given MAF range. The MAF is included in the annotation information associated with the detected variants by the annotating step 104. In a preferred embodiment, the MAF range is [0 10⁻⁶], or MAF is less than or equal to 10⁻⁶. In some embodiments, the MAF range may be [0 0.001], [0 0.002] or [0 0.01]. The MAF ranges may be the same or different for the population databases, such as the 1000 genomes, 5000 exomes and ExAC databases. In filter rule 6, variants found in the UCSC common SNPs database are filtered out. The filter rule set applied to the detected variants may remove the germline variants and retain the somatic variants to produce identified somatic variants, including somatic SNVs and somatic indels.

Some embodiments may include further filtering of the identified somatic mutations to select nonsynonymous SNVs (missense and nonsense mutations) in the exonic region of the panel. Optionally, synonymous SNVs may also be included along with nonsynonymous SNVs. An option to include synonymous SNVs along with nonsynonymous SNVs may be selectable by the user. Further filtering of the somatic indels may select coding sequence somatic indels (frameshift and non-frameshift insertions and deletions). In some embodiments, methods of filtering variants for use with the present teachings may include one or more features described in U.S. Pat. Appl. Publ. No. 2020/0075122, published Mar. 5, 2020, incorporated by reference herein in its entirety.

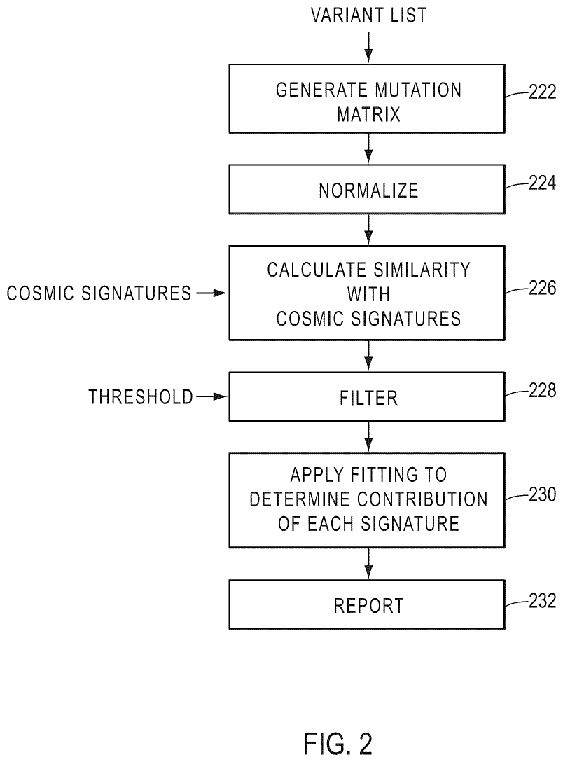

FIG. 2 is a block diagram for detecting mutational signatures from the variant list, in accordance with an embodiment. In the mutation matrix generation step 222, the processor creates a matrix of trinucleotides and trinucleotide counts corresponding to variants on the variant list. A trinucleotide is composed of the variant plus the flanking 5′ and 3′ bases. The processor counts the number of occurrences, or frequency, of each trinucleotide to produce the trinucleotide frequency in the mutation matrix for the panel. In some embodiments, the mutation matrix may include trinucleotide counts for 96 types of triplet mutations. In the normalizing step 224 the trinucleotide frequencies in the mutation matrix may be normalized as follows:
a) For each type of trinucleotide, calculate the ratio y/x, where y is the frequency of the trinucleotide in a reference genome (e.g., hg19) and x is the frequency of the trinucleotide in the portion of the genome covered by the panel. For example, 96 ratios may be calculated for 96 types of trinucleotides.
b) For each trinucleotide in the mutation matrix, multiply the trinucleotide frequency by the corresponding ratio calculated in step a) to form a normalized trinucleotide frequency.
c) Scale the normalized trinucleotide frequencies to values between 0 and 1. For example, the normalized trinucleotide frequencies may be added to form a sum. Each normalized trinucleotide frequency from step b) may be divided by the sum to form a corresponding scaled normalized trinucleotide frequency. The scaled normalized trinucleotide frequencies for the trinucleotides form the normalized mutation matrix for the sample.

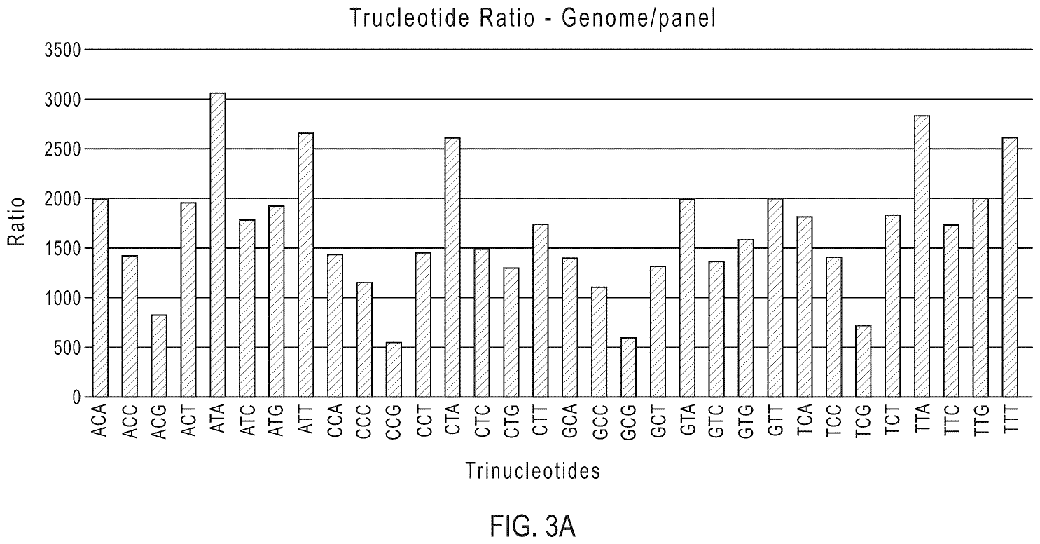

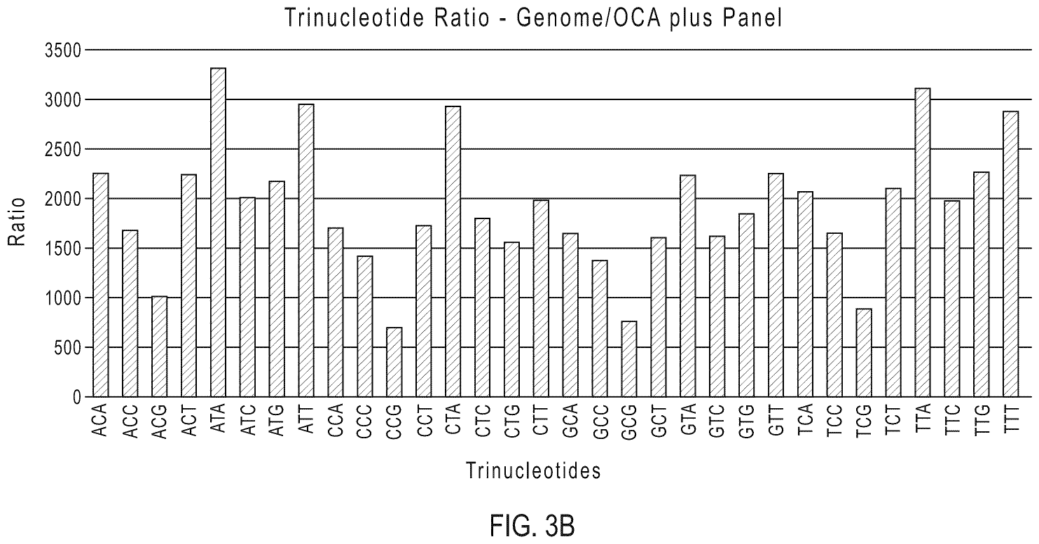

FIG. 3A shows an example of a plot of the ratios of the frequencies of the trinucleotides in an hg19 reference genome to the frequencies of the trinucleotides in the portion of the genome covered by the TML panel. FIG. 3B gives an example of a plot of the ratios of the frequencies of the trinucleotides in an hg19 reference genome to the frequencies of the trinucleotides in the portion of the genome covered by the OCAPlus panel. These examples show the ratios (y/x) for 32 trinucleotides.

Returning to FIG. 2, in step 226, a similarity of the normalized mutation matrix for the sample and a matrix of COSMIC mutational signatures may be calculated. The Catalogue Of Somatic Mutations In Cancer (COSMIC) database is a compendium of mutational signatures (available at www.cancer.sanger.ac.uk/cosmic/signatures). Each COSMIC mutational signature for single base substitutions (SBS) contains 96 triplet mutations and the percentage of single base substitutions for each triplet. For example, the calculation of similarity may be based on the cosine similarity. The cosine similarity measures the cosine of the angle between two vectors in an inner product space. (Manning, C. et al., Introduction to Information Retrieval, Cambridge University Press. 2008. ISBN: 0521865719.) The cosine similarity may be calculated between the normalized mutation matrix and each COSMIC mutational signature in the matrix of COSMIC mutational signatures to form a matrix of similarity values. For example, the cosine similarity values may be determined based on the inner products of the vectors comprising the normalized mutation matrix with the vectors representing the COSMIC mutational signatures. The methods described herein use COSMIC mutational signatures for single base substitutions for exemplary applications. The methods described herein can be applied to other types of variants and corresponding mutational signatures. The methods described herein may use another database, public or private, of mutational signatures.

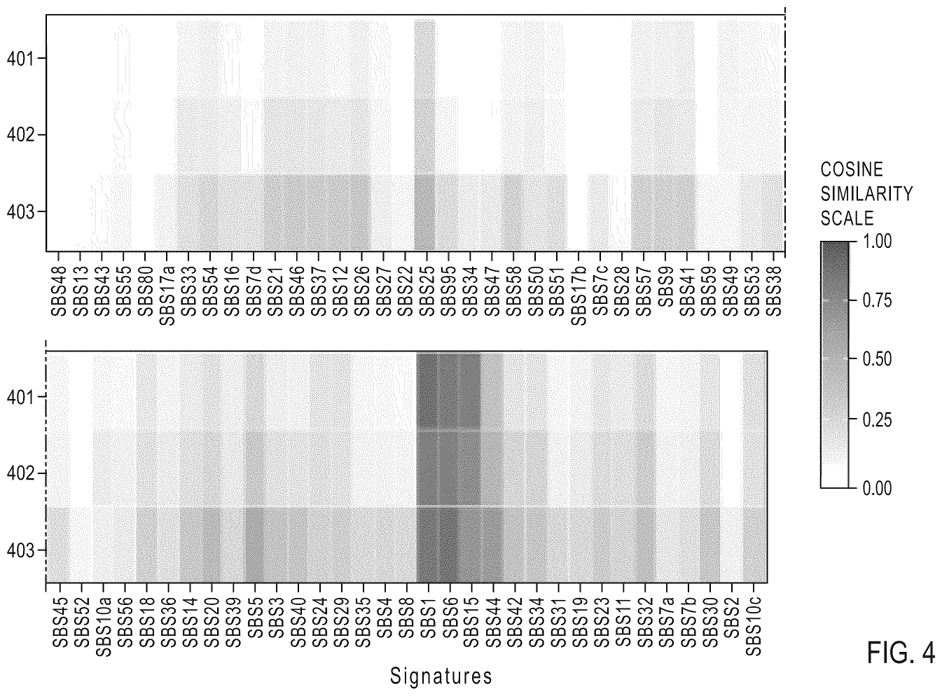

FIG. 4 shows examples of heat maps of the cosine similarity values calculated from sequencing data for whole genome sequencing (WGS), the TML panel with normalization and the TML panel without normalization. The cosine similarities for the whole genome sequencing of the sample are in the heat map row 403. The cosine similarity values for the targeted panel regions calculated using the normalized mutation matrix for the sample from the normalizing step 224 are in the heat map row 402. The cosine similarity values for the targeted panel regions calculated using the trinucleotide frequencies in the mutation matrix without normalization by the normalizing step 224 are in the heat map row 401. The cosine similarity values in heat map row 402 for the targeted panel including the normalizing step 224 are very similar to the cosine similarity values in heat map row 403 for the whole genome sequencing. The cosine similarity values in heat map row 401 for the targeted panel without the normalizing step 224 show more differences with the cosine similarity values in heat map row 403 for the whole genome sequencing. These results show that the mutational signatures predictions made using the targeted panel sequencing data provide the same or very similar results to mutational signature predictions made using the whole genome sequencing data for the same sample. The sample source for this example is Cholangiocarcinoma sample from COSMIC: www.synapse.org/#!Synapse:syn11801870.

In the filtering step 228, each cosine similarity value is compared with a threshold. If the cosine similarity is greater than or equal to the threshold the COSMIC mutational signature may be selected as being present in the sample. A preferred value for the threshold is 0.7. A range of values for the threshold is 0.6 to 0.99. The threshold may be set by the user.

In the fitting step 230, a contribution of each COSMIC mutational signature selected in the filtering step 208 to the normalized mutation matrix is estimated. The normalized mutation matrix for the sample, the COSMIC signature matrix and the list of COSMIC mutational signatures selected in step 228, i.e. those having a cosine similarity greater than or equal to the threshold, may be input to the fitting step 230. The fitting determines a linear combination of the selected COSMIC mutational signatures that optimally reconstructs the normalized mutation matrix for the sample. A weight for each selected COSMIC mutational signature may be found using linear regression or any suitable fitting method. The weight assigned for a given COSMIC mutational signature reflects the proportional contribution of that signature to the sample.

For example, the deconstructSigs package, an extension for the R programming language, may be applied to determine the weights. The deconstructSigs package applies an iterative approach to calculate weights that minimize the sum-squared error (SSE) between the normalized mutation matrix for the sample and the sum of the weighted COSMIC mutational signatures. The deconstructSigs package is available on the Comprehensive R Archive Network (CRAN, www.cran.r-project.org/). (See Rosenthal, R. et al., deconstructSigs: delineating mutational processes in single tumors distinguishes DNA repair deficiencies and patterns of carcinoma evolution, GenomeBiol 17, 31 (2016), www.doi.org/10.1186/s13059-016-0893-4).

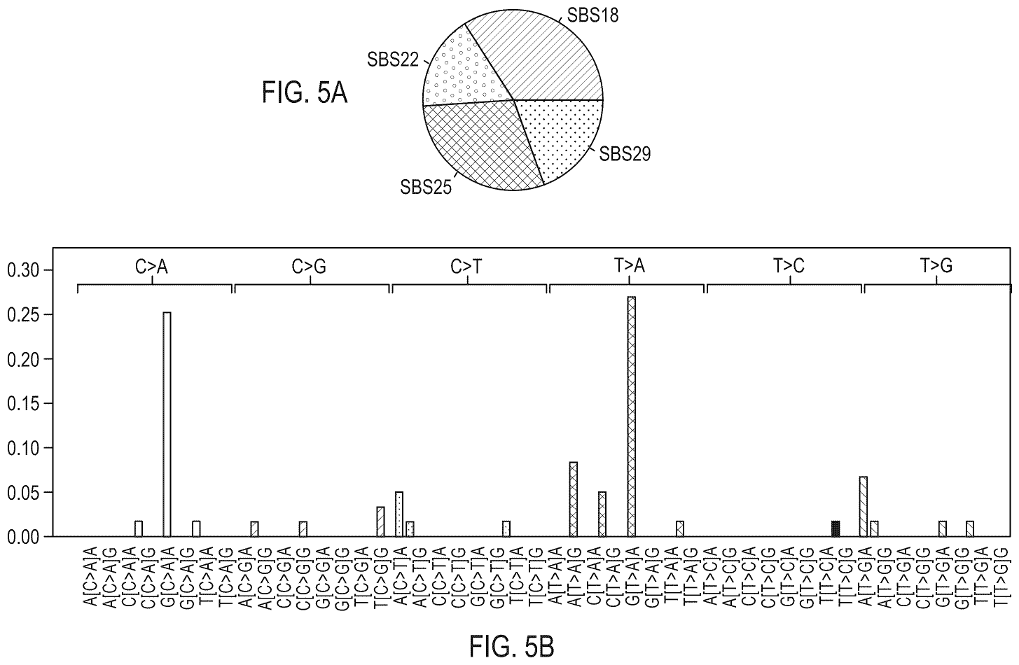

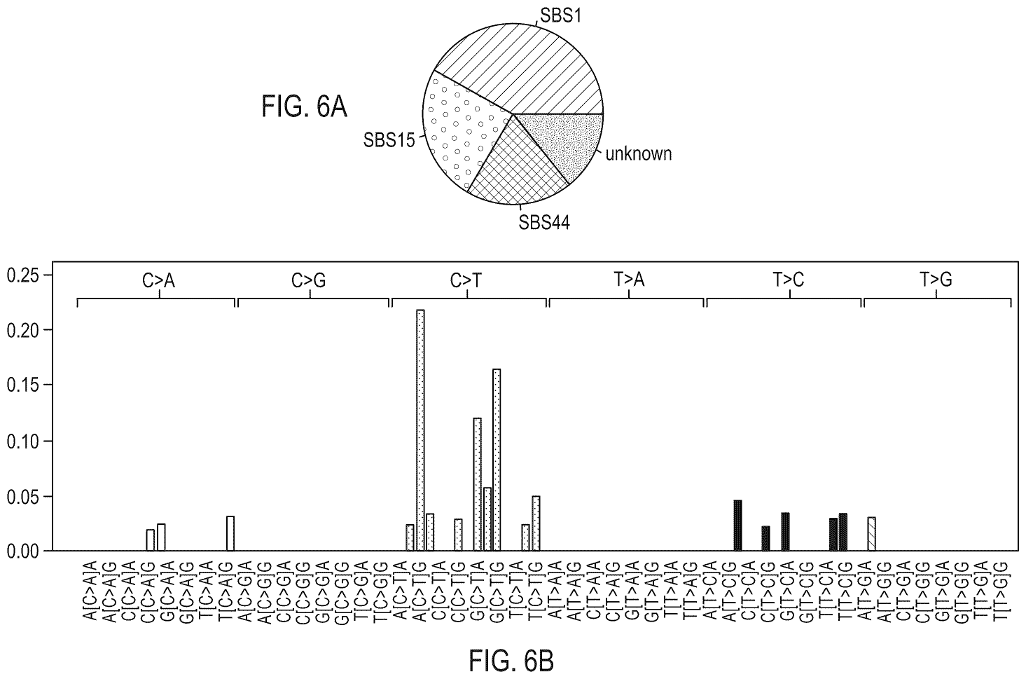

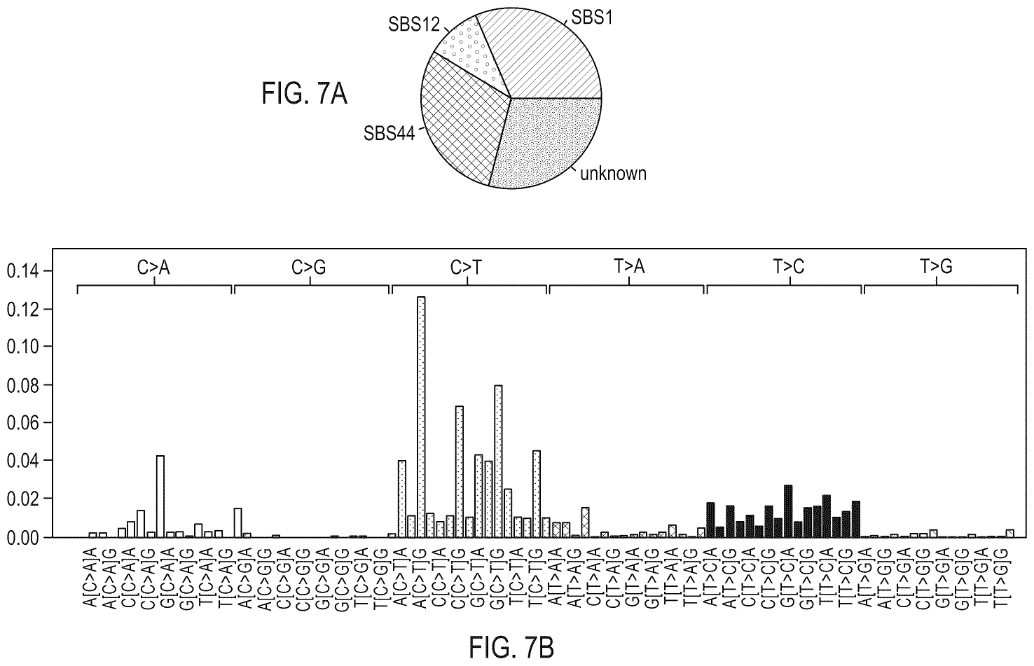

FIG. 5A shows an example of a pie chart showing the contributions of the selected COSMIC mutational signatures to the mutation matrix for the TML targeted panel without the normalizing step 224. The “SBS” labels are the identifiers for the COSMIC mutational signatures (www.cancer.sanger.ac.uk/cosmic/signatures/index.tt). FIG. 5B is an example of a plot of the trinucleotide frequencies for the TML targeted panel without the normalizing step 224. FIG. 6A shows an example of a pie chart showing the contributions of the selected COSMIC mutational signatures to the normalized mutation matrix for the TML targeted panel with the normalizing step 224. FIG. 6B is an example of a plot of the trinucleotide frequencies for the TML targeted panel with the normalizing step 224. FIG. 7A shows an example of a pie chart showing the contributions of the selected COSMIC mutational signatures using whole genome sequencing data. FIG. 7B is an example of a plot of the trinucleotide frequencies for the whole genome sequencing data. Comparison of these results show that FIGS. 6A and 6B for the targeted panel with the normalizing step 224 are more similar to the results for the whole genome sequencing data of FIGS. 7A and 7B than are the results in FIGS. SA and 5B for the targeted panel without the normalizing step 224.

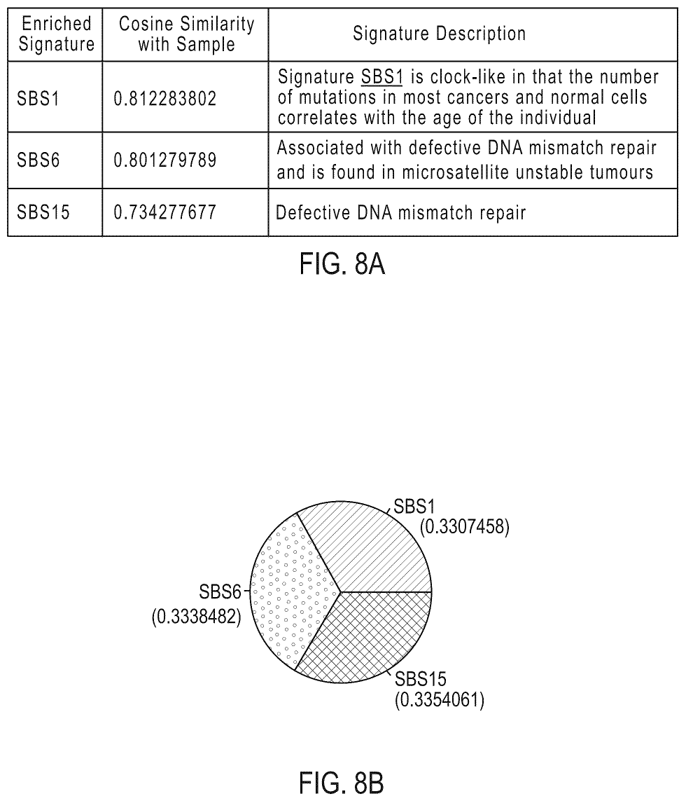

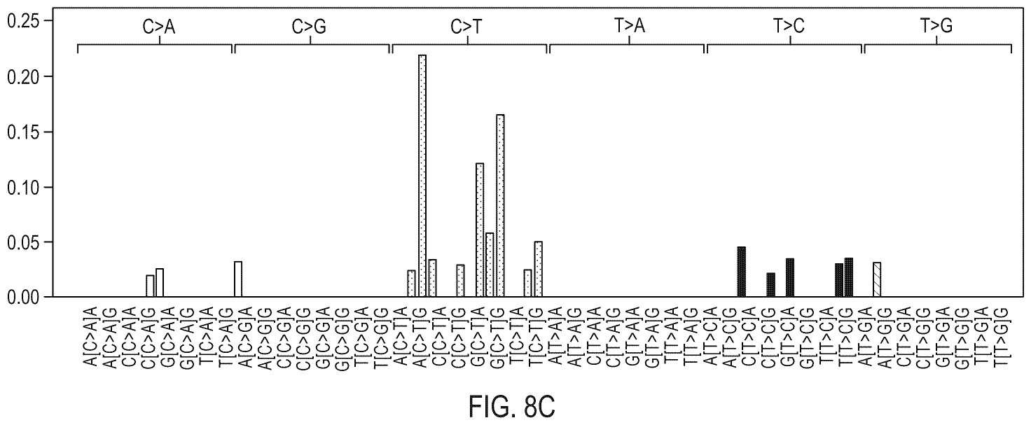

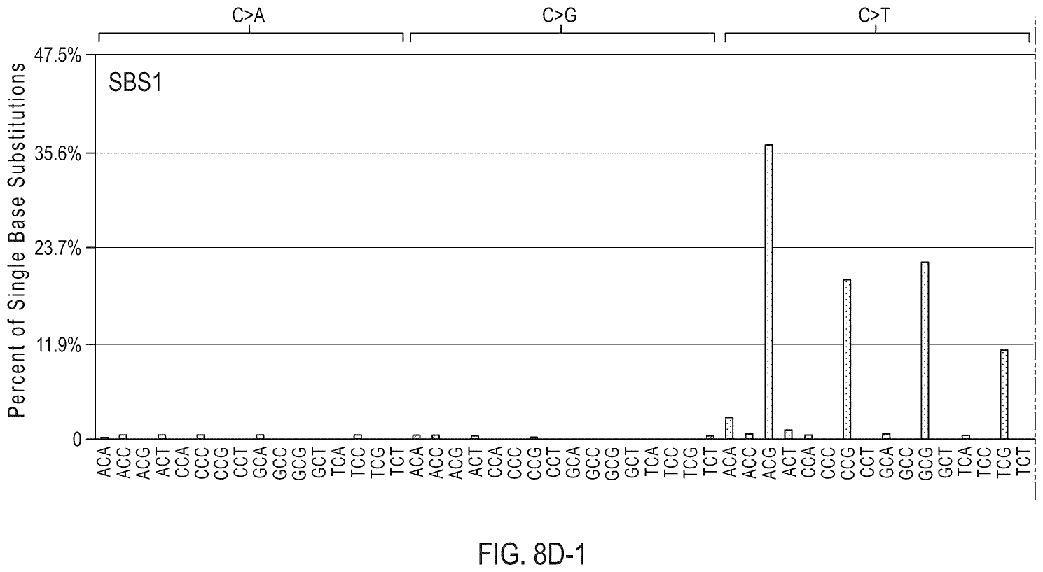

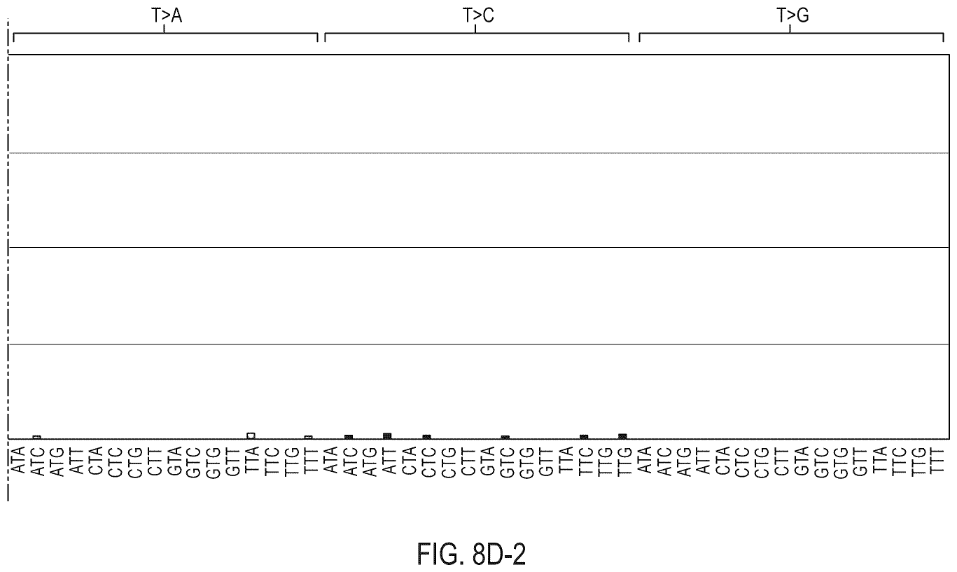

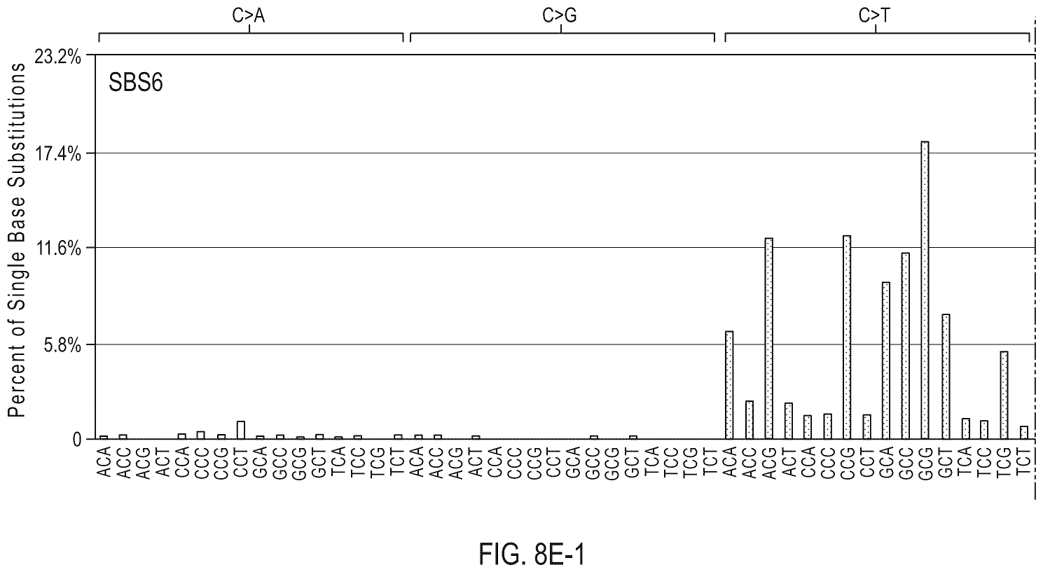

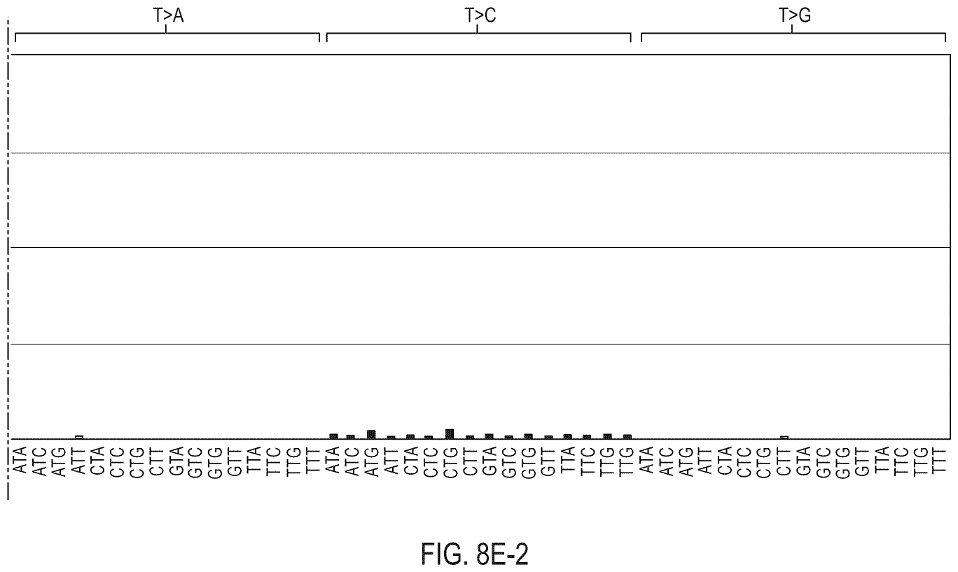

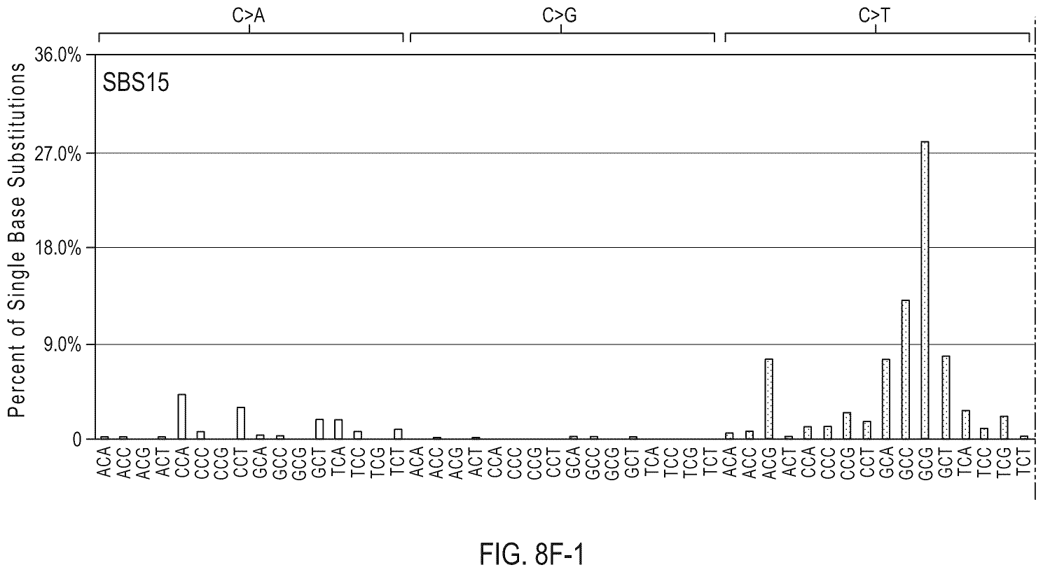

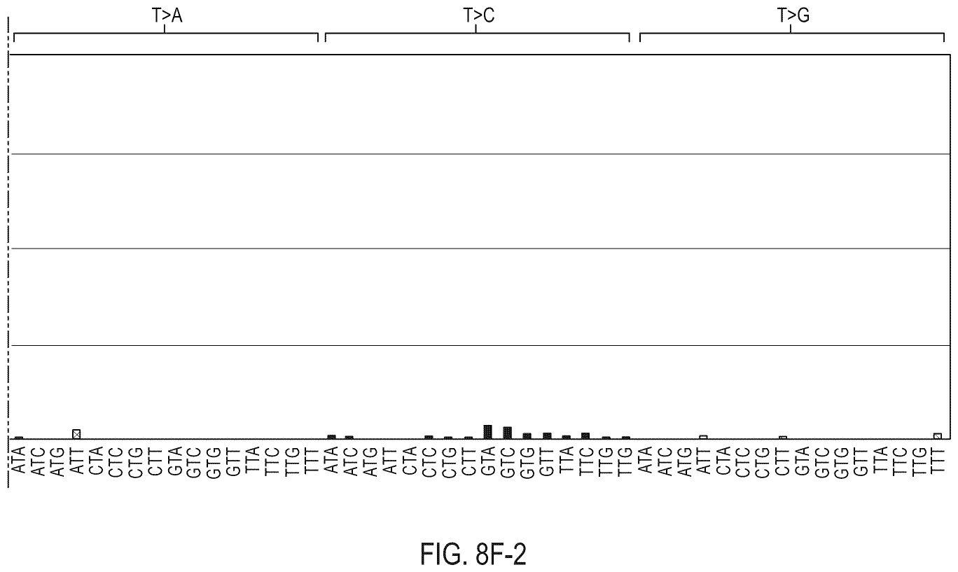

Returning to FIG. 2, the report step 220, may provide results in a display for the user. FIGS. 8A-8F show examples of results that may be included in a display for the user.
Table with list of the selected COSMIC mutational signatures and description of the signatures in terms of the etiology. (FIG. 8A)
Pie chart reflecting the proportional contribution of the selected COSMIC mutational signatures in the sample. (FIG. 8B)
Bar graph representing the normalized mutation matrix for the sample. (FIG. 8C)
Bar graphs representing the selected COSMIC mutational signatures. (FIGS. 8D-1, 8D-2, 8E-1, 8E-2, 8F-1 and 8F-2)

Data sets tested for mutational signature detected using a targeted panel, Oncomine Tumor Mutation Load Assay (TML), are shown in TABLE 3.

TABLE 3

| | | NUMBER OF SAMPLES |
|---|---|---|
| | NUMBER OF | WITH AT LEAST ONE |
| | RUNS | SIGNATURE ≥ 0.7 |
| TEST SET 1 | 550 | 144 |
| TEST SET 2 | 1043 | 462 |
| TOTAL | 1,593 | 606 |

The data sets represent a variety of solid tumors showing mutational signatures related to UV damage, tobacco damage and MMR (mismatch repair), as shown in the results below.

TABLE 4 shows results for COSMIC mutational signatures related to UV damage. The counts show the number of samples where the cosine similarity is greater than or equal to 0.7, indicating detection of the corresponding mutational signature in the sample.

TABLE 4

| COSMIC MUTATIONAL | TEST SET | TEST SET |
|---|---|---|
| SIGNATURE ID | 1 COUNTS | 2 COUNTS |
| SBS7a | 44 | 50 |
| SBS7b | 33 | 130 |
| SBS7c | 0 | 0 |
| SBS7d | 2 | 0 |
| SBS38 | 2 | 0 |

TABLE 5 shows results for COSMIC mutational signatures related to tobacco damage. The counts show the number of samples where the cosine similarity is greater than or equal to 0.7, indicating detection of the corresponding mutational signature in the sample.

TABLE 5

| COSMIC MUTATIONAL | TEST SET | TEST SET |
|---|---|---|
| SIGNATURE ID | 1 COUNTS | 2 COUNTS |
| SBS4 | 5 | 8 |
| SBS29 | 2 | 0 |

TABLE 6 shows results for COSMIC mutational signatures related to mismatch repair (MMR). The counts show the number of samples where the cosine similarity is greater than or equal to 0.7, indicating detection of the corresponding mutational signature in the sample.

TABLE 6

| COSMIC MUTATIONAL | TEST SET | TEST SET |
|---|---|---|
| SIGNATURE ID | 1 COUNTS | 2 COUNTS |
| SBS6 | 1 | 1 |
| SBS14 | 1 | 0 |
| SBS15 | 2 | 8 |
| SBS20 | 3 | 0 |
| SBS21 | 0 | 10 |
| SBS44 | 13 | 26 |

TABLE 7 shows results for COSMIC mutational signatures related to other types of repair. The counts show the number of samples where the cosine similarity is greater than or equal to 0.7, indicating detection of the corresponding mutational signature in the sample.

TABLE 7

| COSMIC MUTATIONAL | TEST SET | TEST SET |
|---|---|---|
| SIGNATURE ID | 1 COUNTS | 2 COUNTS |
| SBS30 | 69 | 334 |
| SBS20 | 2 | 8 |
| SBS36 | 5 | 3 |

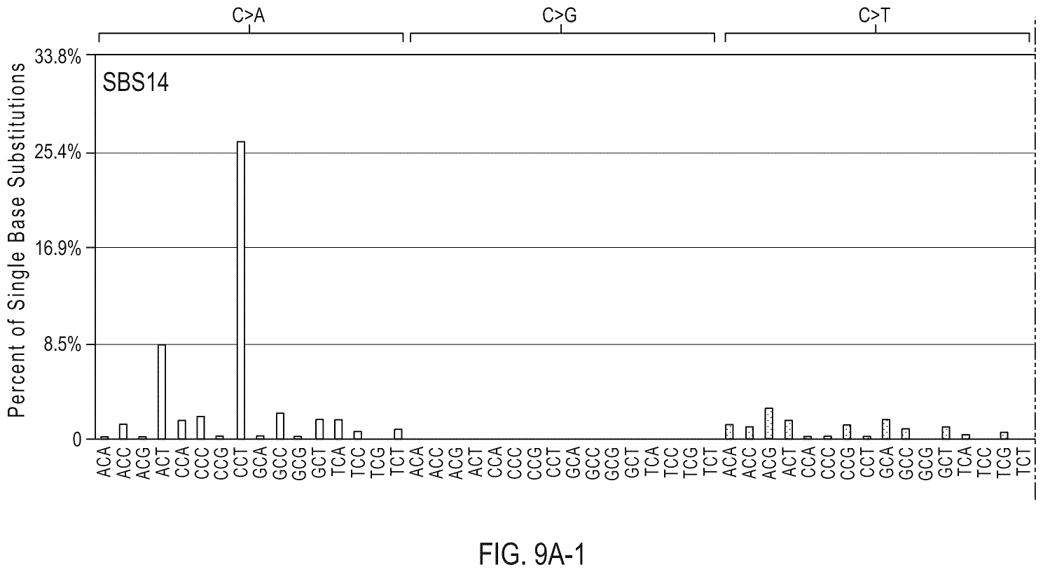

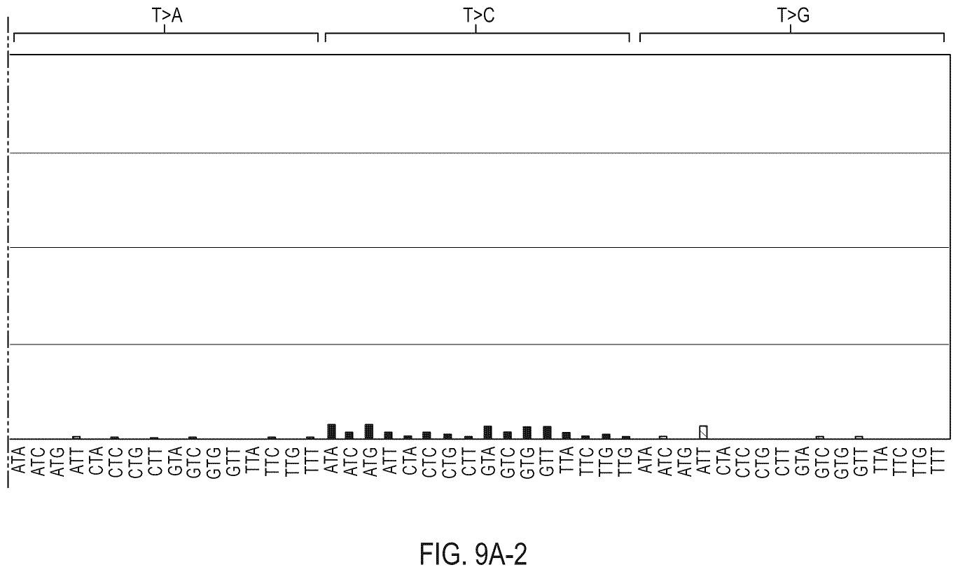

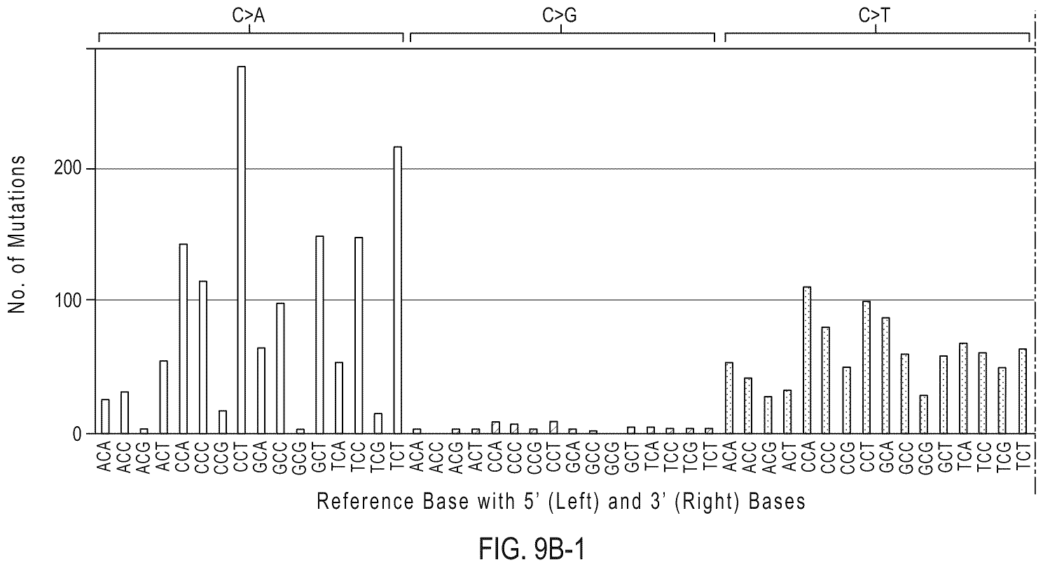

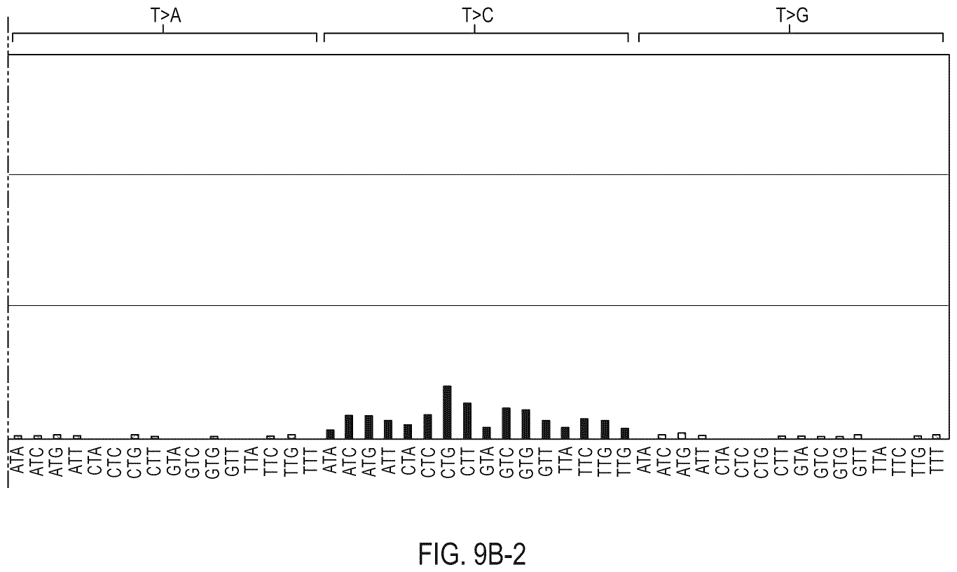

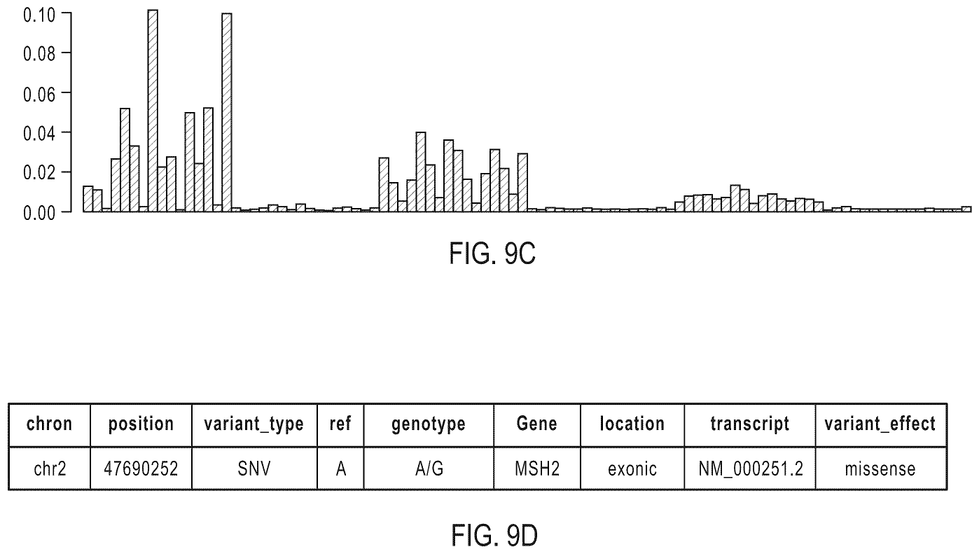

Some mutational signatures are caused by deficiency in various mismatch repair enzymes. These are often caused by somatic and/or germline mutations in specific gene. The following shows that it is possible to detect a mutation in a gene, and also to detect the resulting mutational signature that it causes. FIGS. 9A to 9D, FIGS. 10A to 10D, FIGS. 11A to 11D, FIGS. 12A to 12D, and FIGS. 13A to 13F show several such examples of results generated using the TML targeted panel.

FIGS. 9A to 9D show results for a sample with high cosine similarity=0.8046 for MMR signature SBS14 and an MMR gene MSH2 mutation. FIG. 9A-1 to 9A-2 shows the bar graph representing the selected COSMIC mutational signature SBS14. FIG. 9B-1 to 9B-2 shows a plot of the trinucleotide frequencies for the sample before the normalizing step 224. FIG. 9C shows of a plot of the trinucleotide frequencies for the sample after the normalizing step 224. FIG. 9D gives the details of the detected variant.

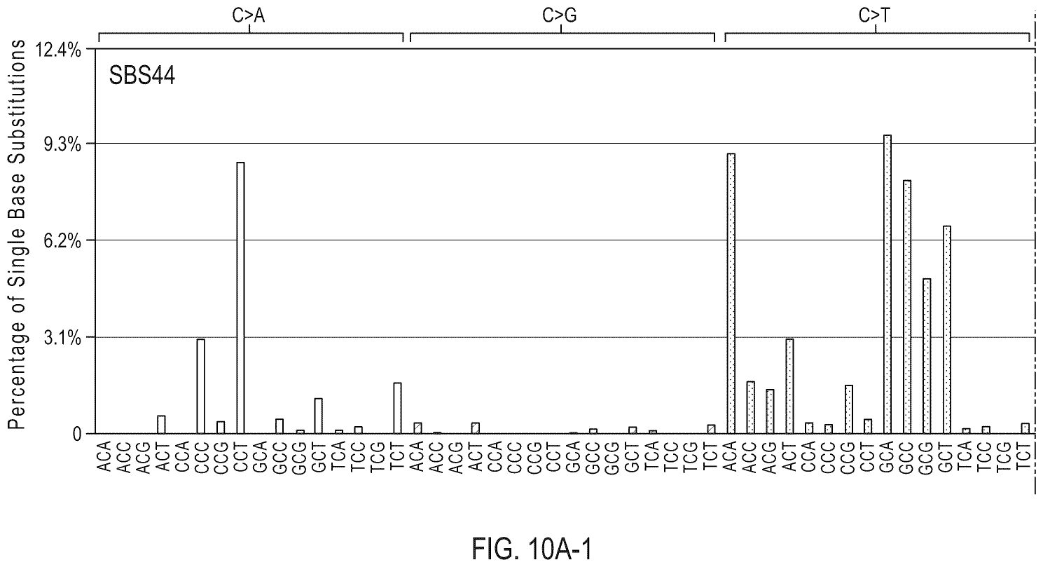

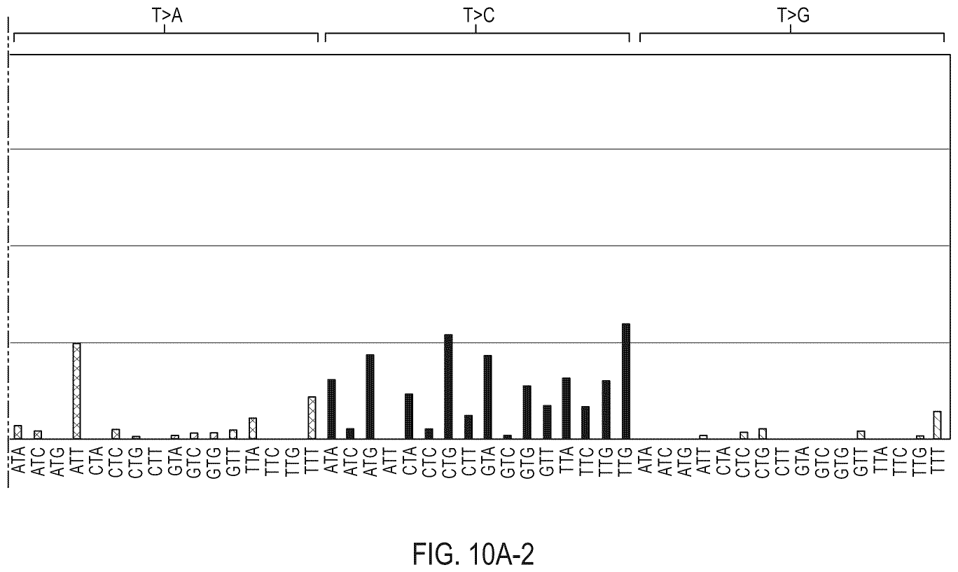

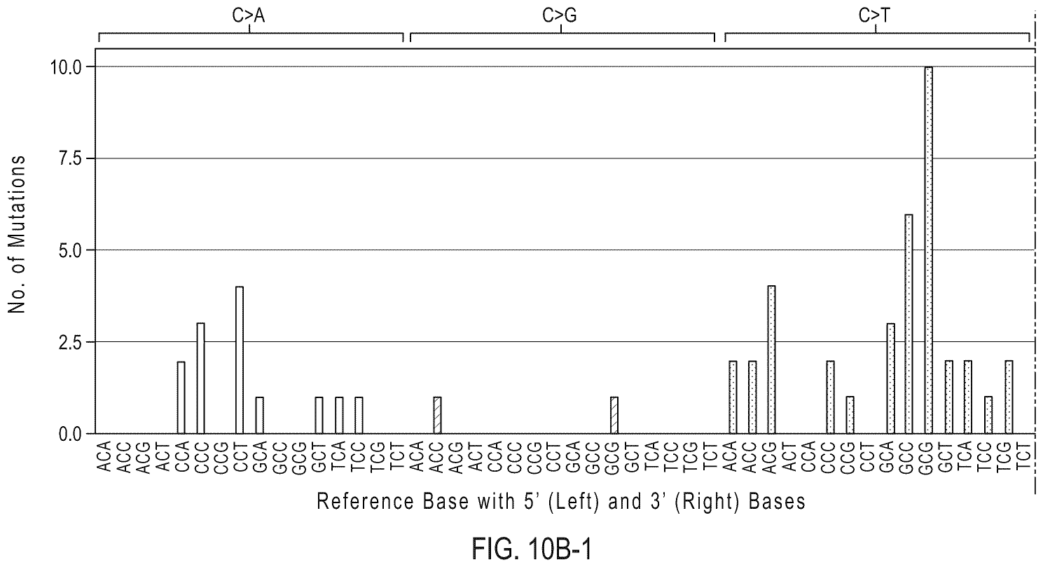

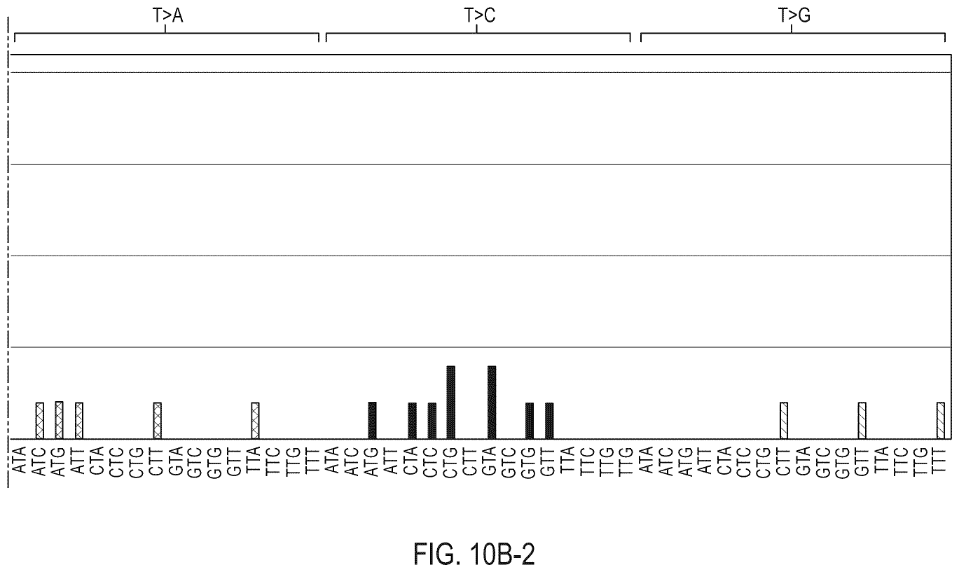

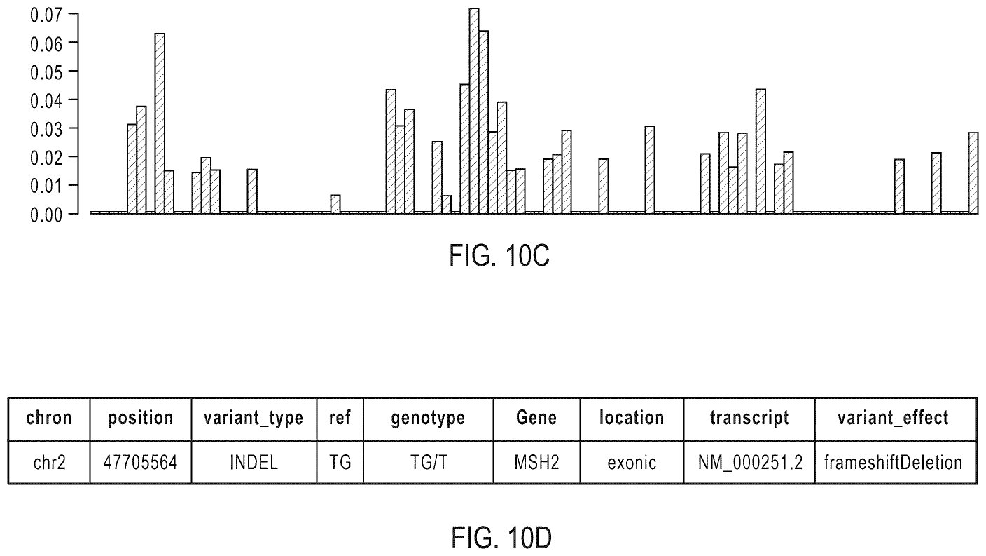

FIGS. 10A to 10D show results for a sample with high cosine similarity=0.7737 for MMR signature SBS44 and an MMR gene MSH2 mutation. FIG. 10A-1 to 10A-2 shows the bar graph representing the selected COSMIC mutational signature SBS44. FIG. 10B-1 to 10B-2 shows of a plot of the trinucleotide frequencies for the sample before the normalizing step 224. FIG. 10C shows of a plot of the trinucleotide frequencies for the sample after the normalizing step 224. FIG. 10D gives the details of the detected variant.

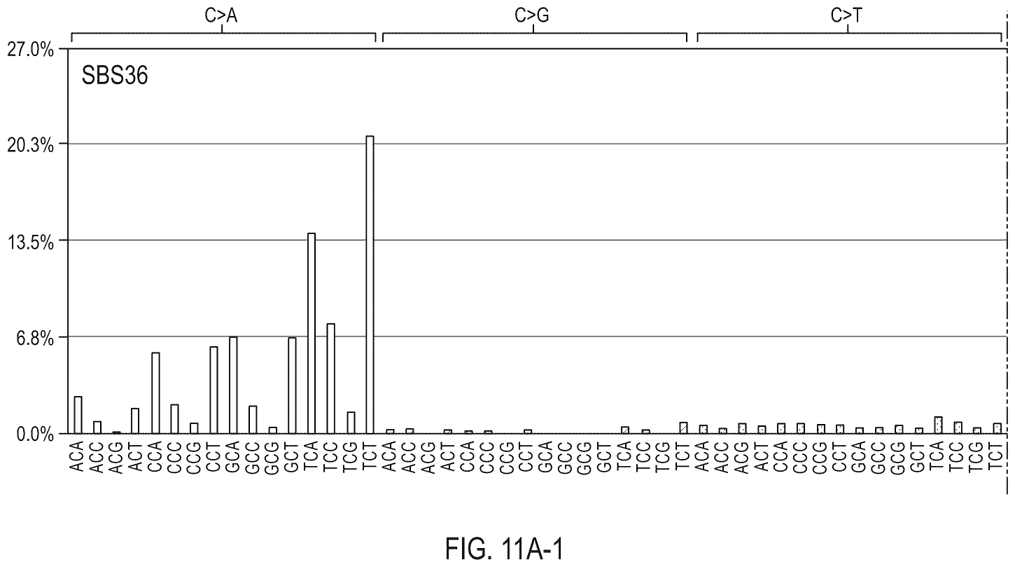

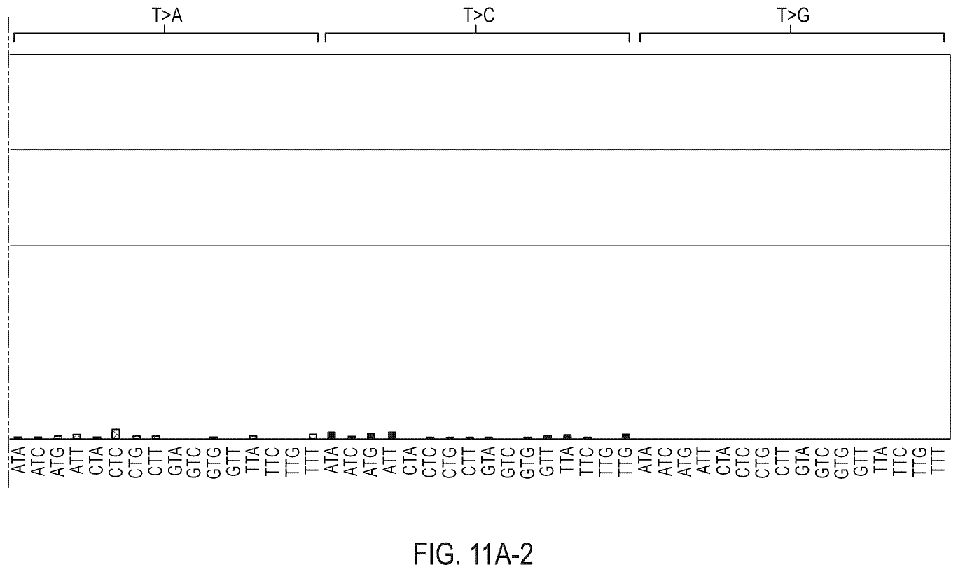

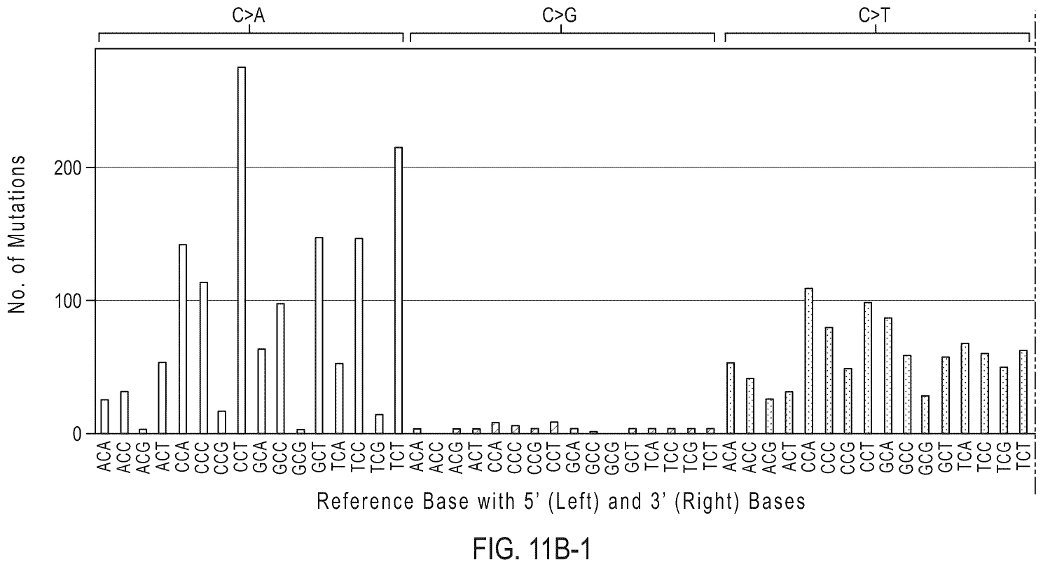

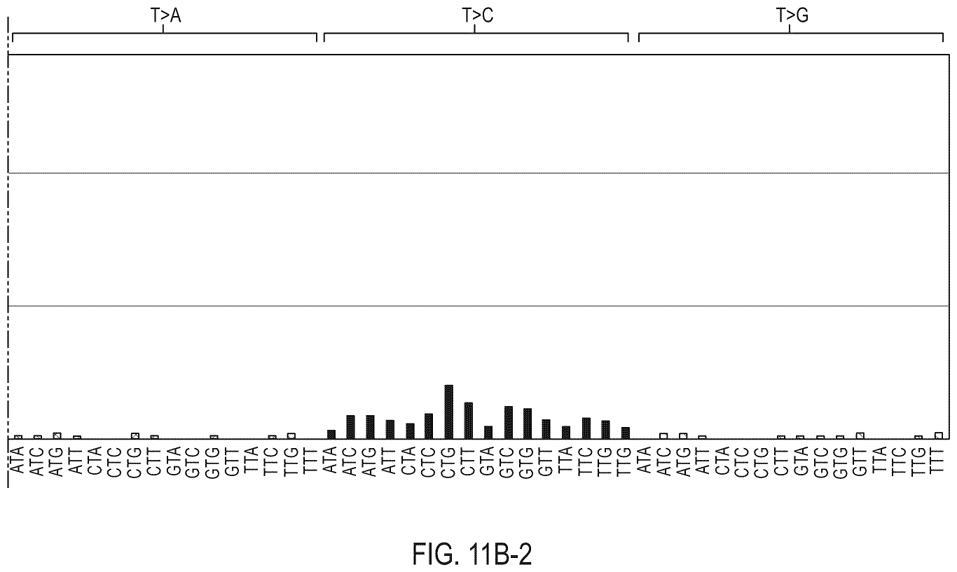

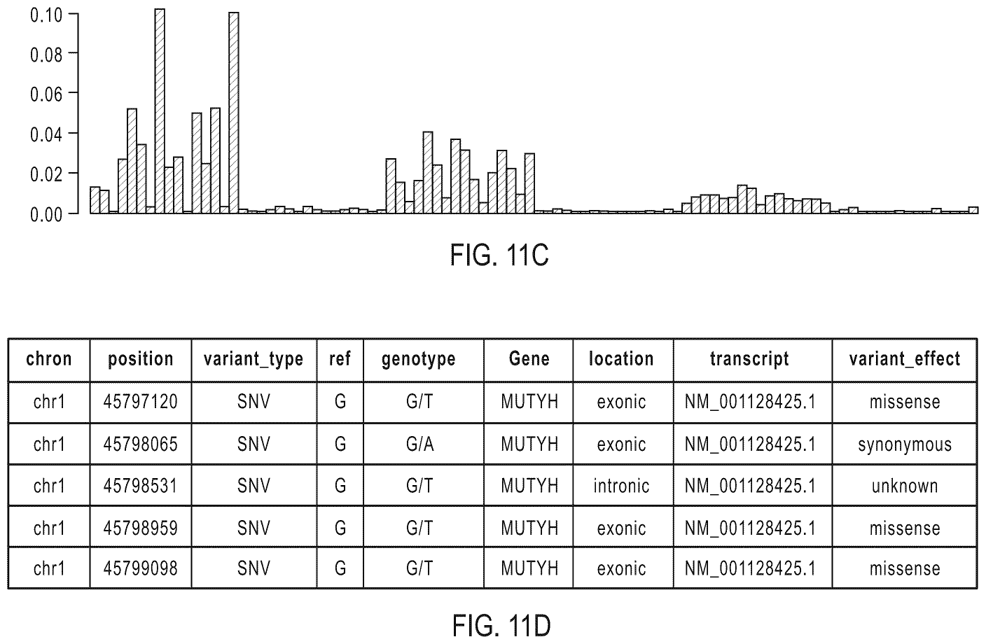

FIGS. 11A to 11D show results for a sample with cosine similarity=0.7691 for SBS36 signature and MUTYH mutations. FIG. 11A-1 to 11A-2 shows the bar graph representing the selected COSMIC mutational signature SBS36. FIG. 11B-1 to 11B-2 shows of a plot of the trinucleotide frequencies for the sample before the normalizing step 224. FIG. 11C shows of a plot of the trinucleotide frequencies for the sample after the normalizing step 224. FIG. 11D gives the details of the detected variants.

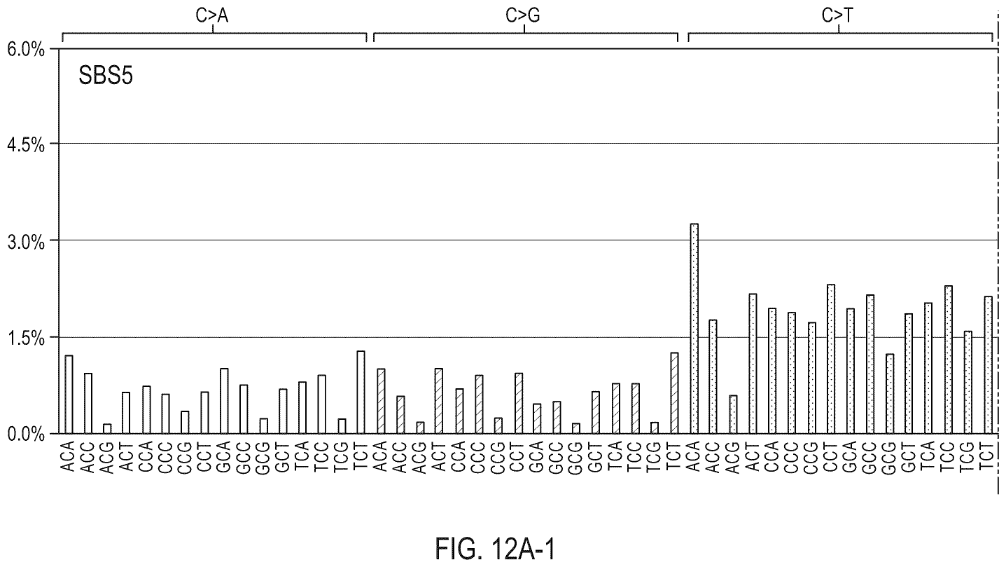

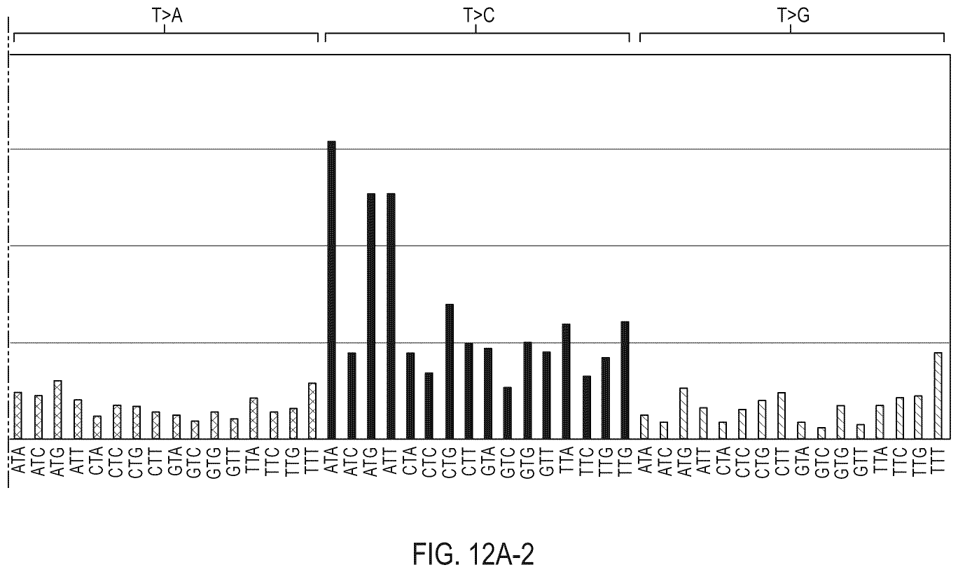

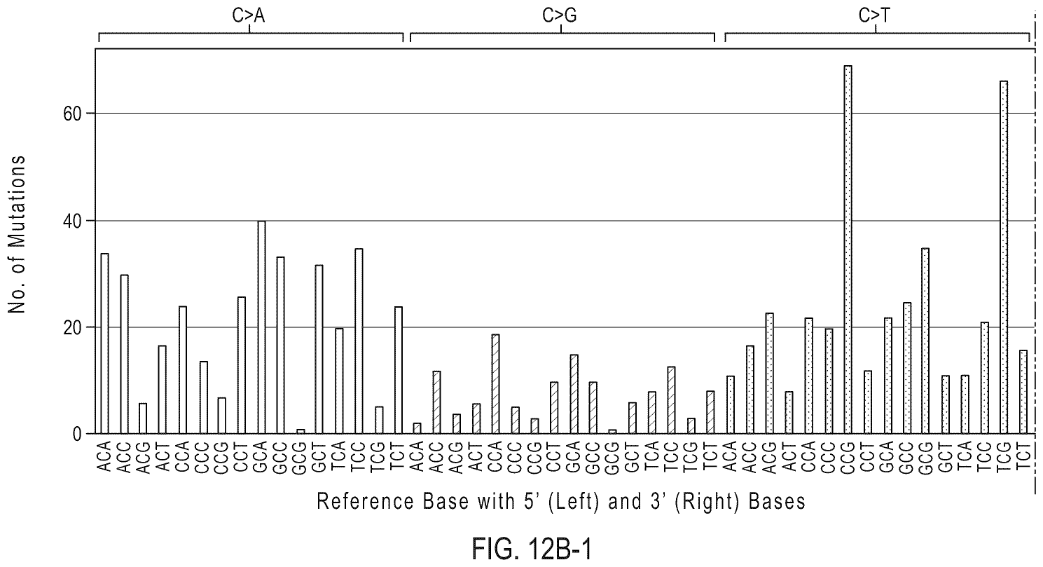

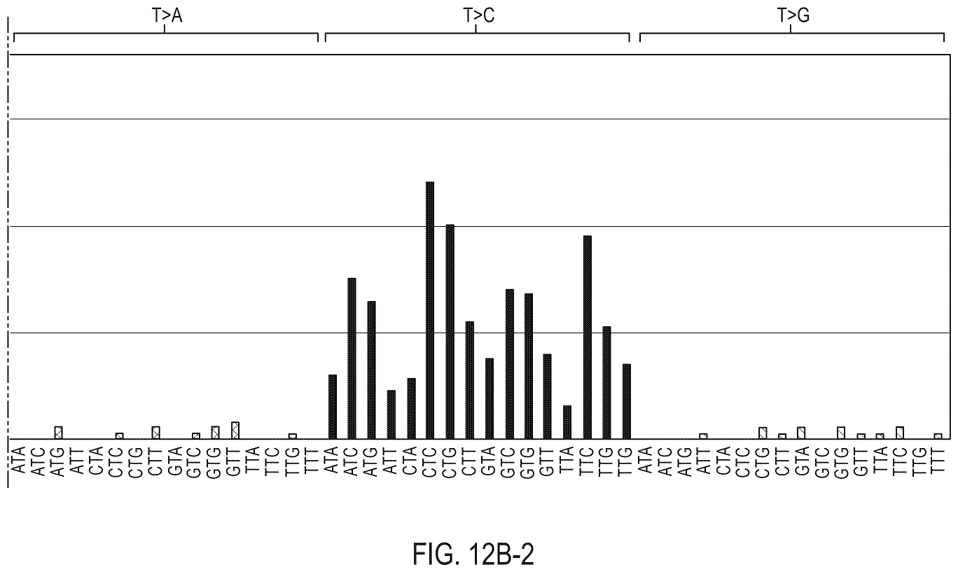

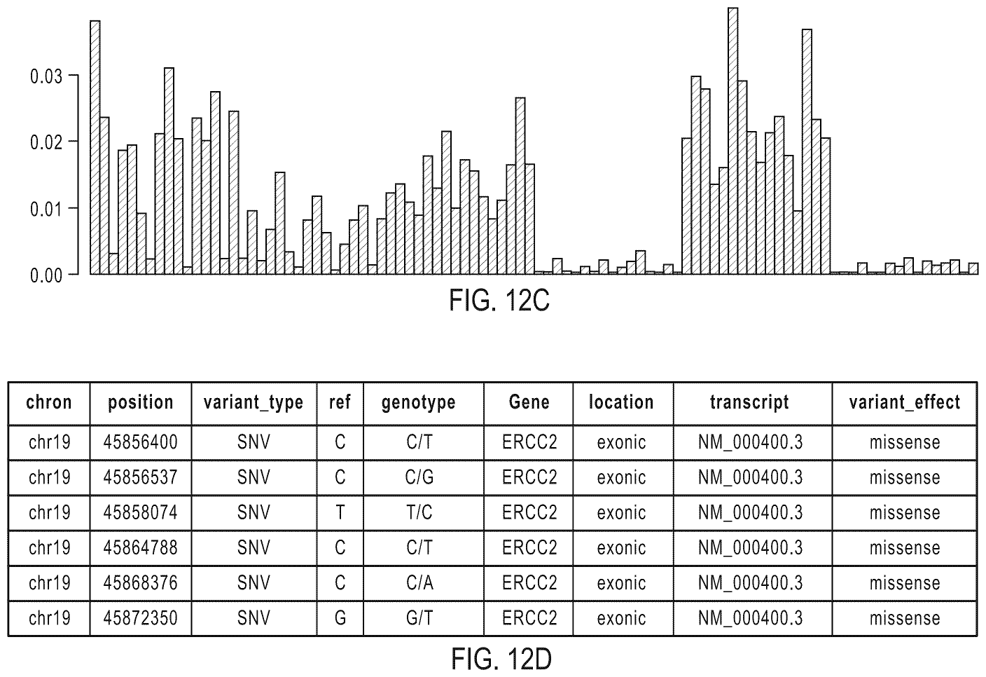

FIGS. 12A to 12D show results for a sample with cosine similarity=0.7634 for SBS5 signature and expected ERCC2 gene mutations. FIG. 12A-1 to 12A-2 shows the bar graph representing the selected COSMIC mutational signature SBS5. FIG. 12B-1 to 12B-2 shows of a plot of the trinucleotide frequencies for the sample before the normalizing step 224. FIG. 12C shows of a plot of the trinucleotide frequencies for the sample after the normalizing step 224. FIG. 12D gives the details of the detected variants.

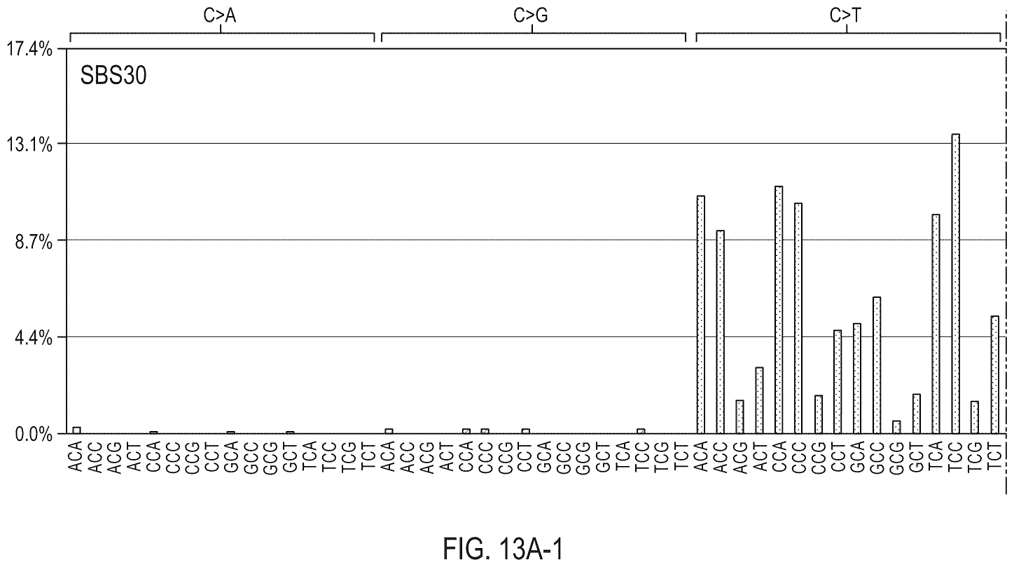

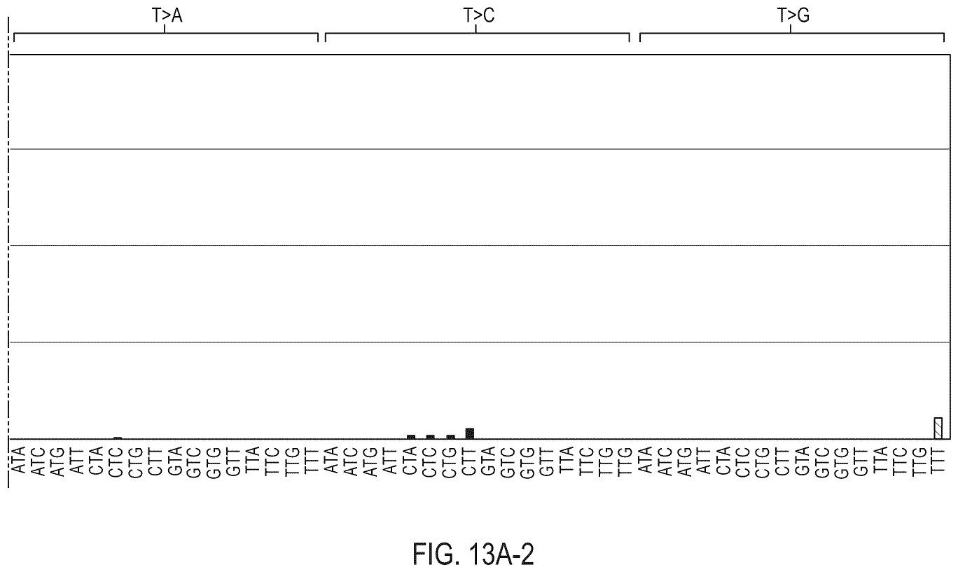

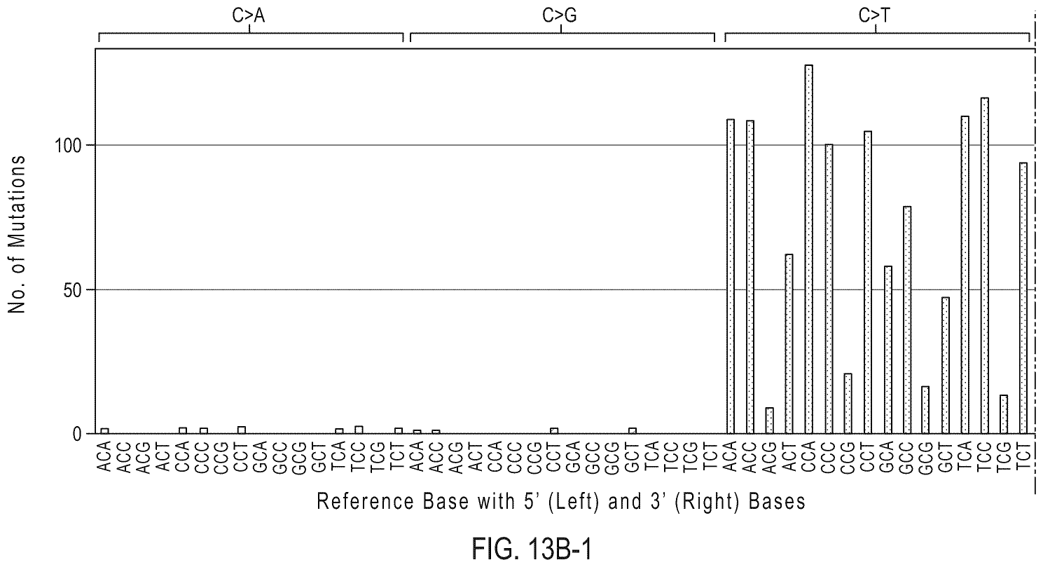

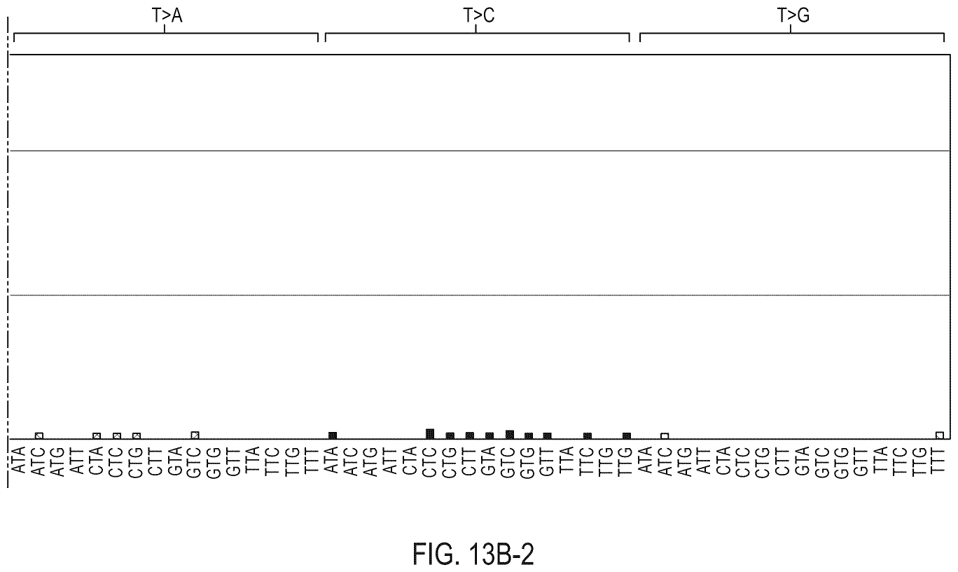

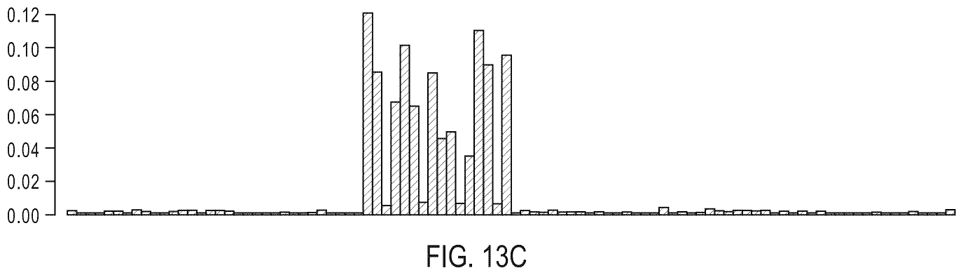

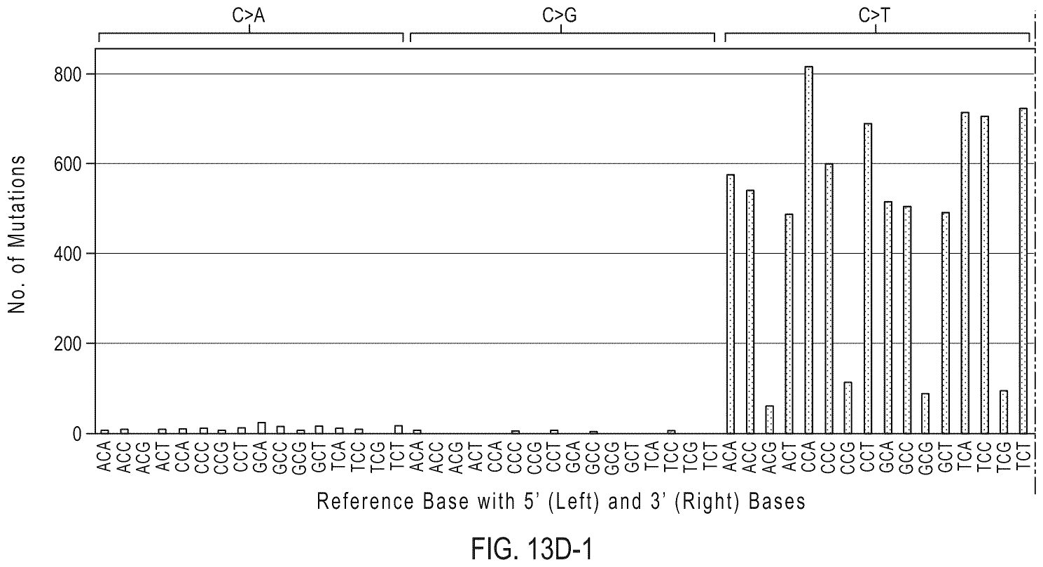

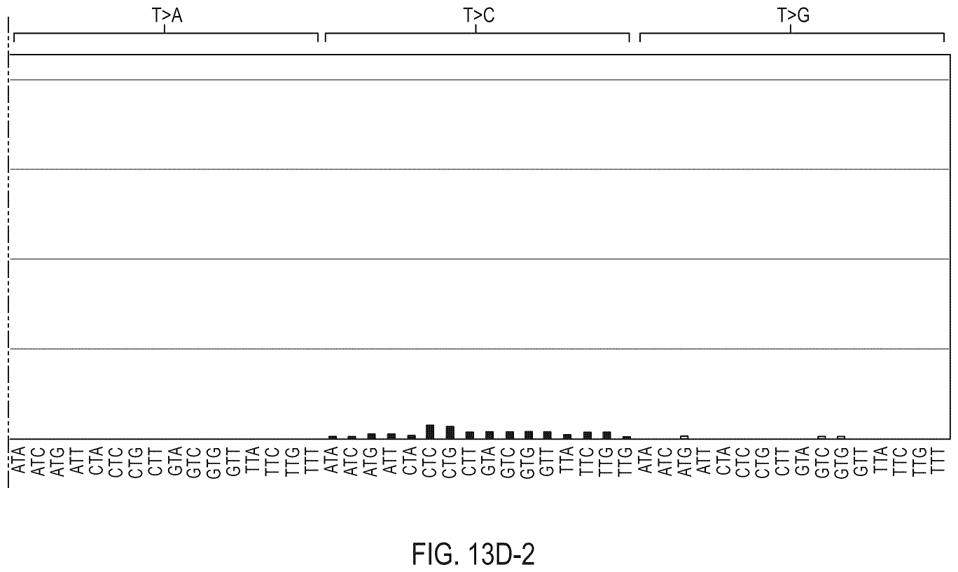

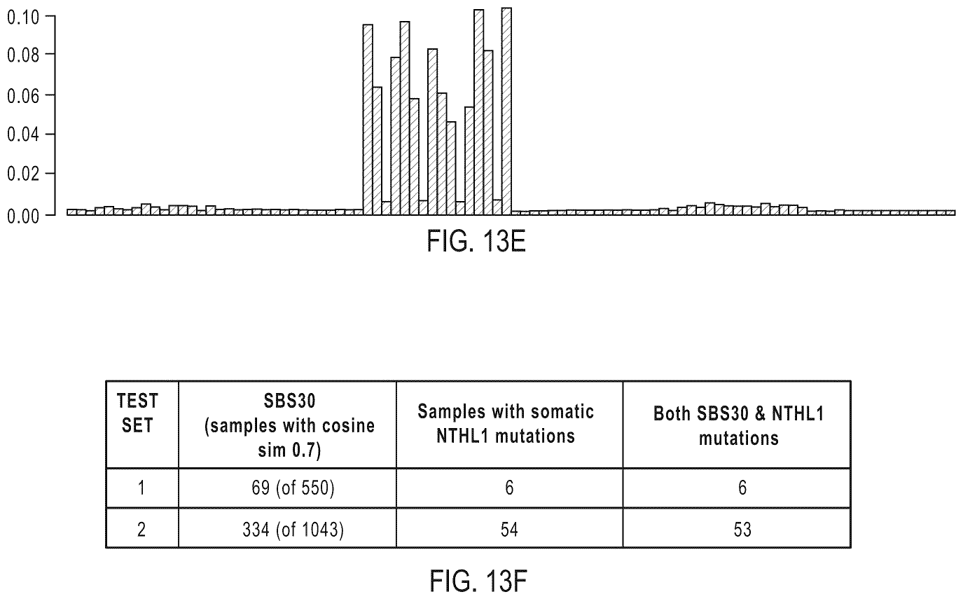

FIGS. 13A to 13F show results for two samples for SBS30 base excision repair signature and expected NTHLI mutations. FIG. 13A-1 to 13A-2 shows the bar graph representing the selected COSMIC mutational signature SBS30. FIG. 13B-1 to 13B-2 shows of a plot of the trinucleotide frequencies for the first sample before the normalizing step 224. FIG. 13C shows of a plot of the trinucleotide frequencies for the first sample after the normalizing step 224. FIG. 13D-1 to 13D-2 shows of a plot of the trinucleotide frequencies for the second sample before the normalizing step 224. FIG. 13E shows of a plot of the trinucleotide frequencies for the second sample after the normalizing step 224. FIG. 13F gives the results of the detections of the SBS30 signature and the NTHLI variants for test set 1 and test set 2.

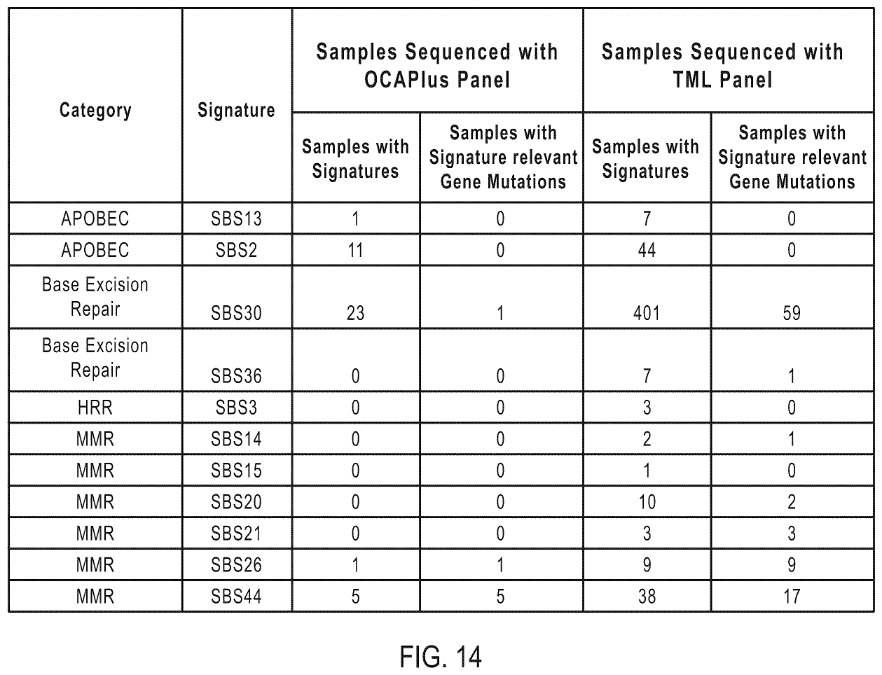

FIG. 14 is a table of results showing the prevalence of base excision repair, MMR, APOBEC and HRR signatures in data obtained from samples sequenced using the OCAPlus panel and data obtained from samples sequenced using the TML panel.

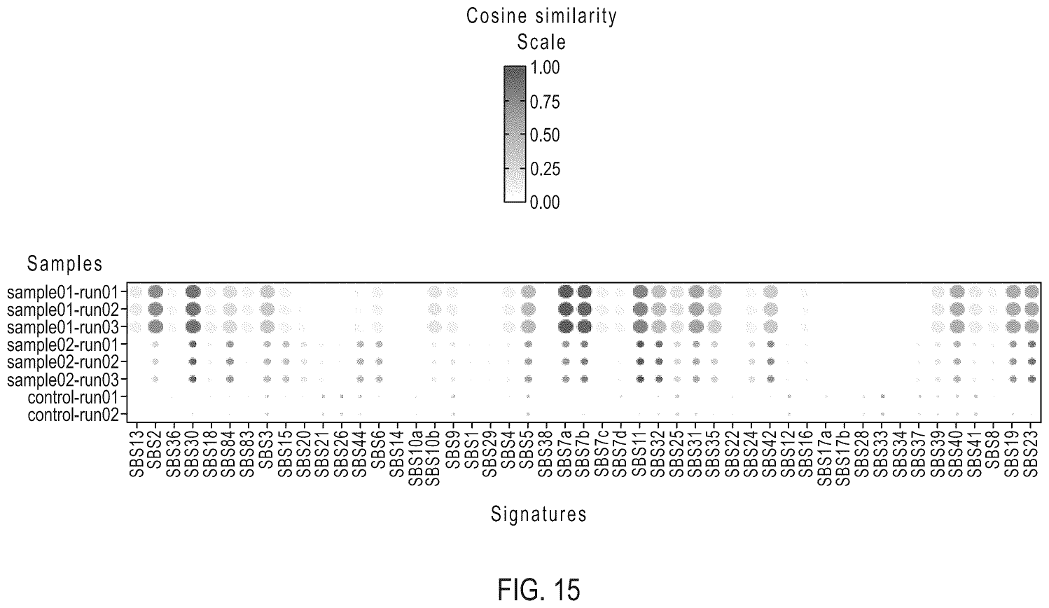

FIG. 15 gives results of mutational signature predictions for two tumor samples and a control sample. The method for detecting mutational signatures was run three times on each of the two tumor samples and two times on the control sample. The sizes of the bubbles in the chart indicates the number of variants determined for the tumor samples and control sample. The shading indicates the cosine similarity values determined for the tumor samples and control sample. These results show that the detections of mutational signatures were reproducible for the tumor samples and the control sample.

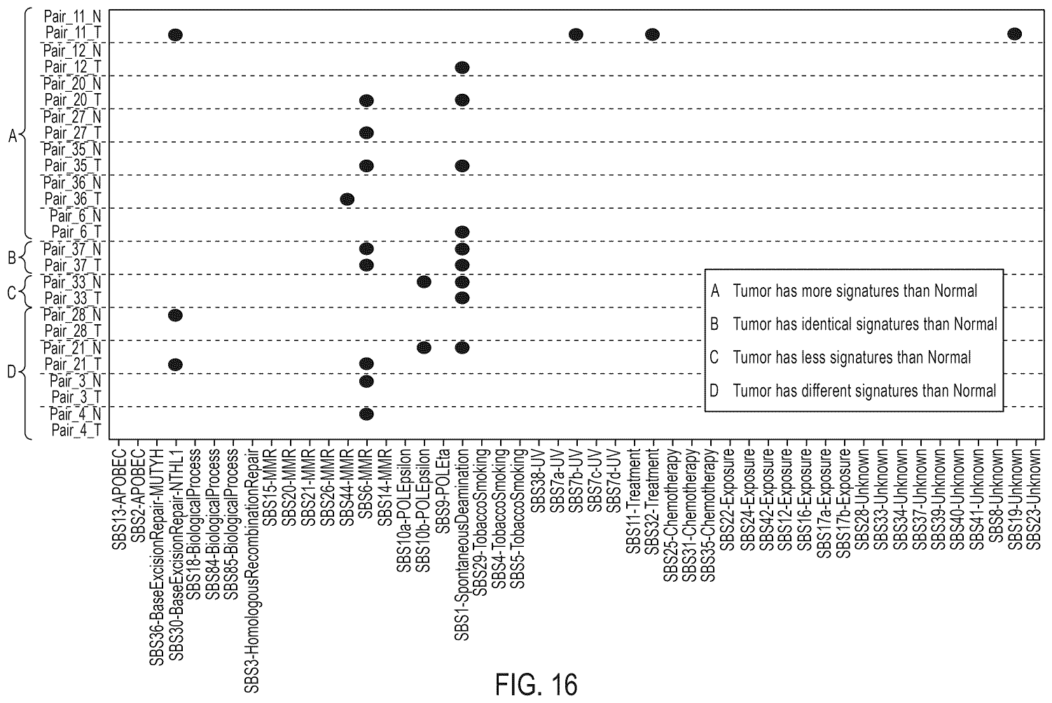

FIG. 16 shows an example of results that compare detections of mutational signatures in tumor samples and matched normal samples. The mutational signatures were identified in data obtained from tumor/normal sample pairs sequenced using the OCAPlus panel. The pairs are displayed along the y-axis, where “T” indicates the tumor sample and “N” indicates the matched normal sample. The mutational signature identifiers are displayed along the x-axis. A dot indicates a mutational signature along the x-axis was detected for a sample along the y-axis. The shading of the pairs along the y-axis indicate whether the tumor sample has more, same, less, or different detected mutational signatures the matched normal sample. These results show that the tumor samples generally have more mutational signatures than their matched normal samples.

The targeted panel and method for detecting mutational signatures described herein provide improvements to the technology over WES or WGS based technology. For WES, 30 Mb of the tumor genome would be covered. In comparison, the targeted panel covers a smaller portion of the tumor genome, e.g. 1.65 Mb by the Oncomine Tumor Mutation Load Assay and 1.4 Mb by the Oncomine Comprehensive Assay Plus. This is especially advantageous because of the limited availability of DNA in FFPE samples and the higher success rates of targeted amplicon-based sequencing. The data resulting from the nucleic acid sequence reads of the 30 Mb would require computations to detect variants and storage. For sequence assembly, methods must be able to assemble and/or map a large number of sequence reads efficiently, such as by minimizing use of computational resources. For example, the sequencing of a human genome can result in tens or hundreds of millions of reads that need to be assembled before they can be further analyzed. Computer processing of the nucleic acid sequence reads from targeted sequencing reduces computational requirements and memory requirements versus processing for WES or WGS data. Processing the sequencing data from these assays would require substantially fewer computations for detecting variants and substantially less memory required for storage of the nucleic acid sequence reads and variant data. As shown in the results of FIG. 4, the mutational signatures predictions made using the targeted panel sequencing data provide the same or very similar results to mutational signature predictions made using the WGS data for the same sample.

According to an exemplary embodiment, there is provided a method of analyzing a tumor sample genome for a mutational signature, including the following steps: (1) amplifying nucleic acid sequences at targeted locations in the tumor sample genome by a targeted panel to generate a plurality of nucleic acid sequence reads; (2) detecting variants in the plurality of nucleic acid sequence reads to produce a plurality of variants: (3) generating a set of trinucleotides by appending a flanking 5′ base and a flanking 3′ base to each variant: (4) determining a frequency of each trinucleotide in the set of trinucleotides to form a mutation matrix; (5) determining a cosine similarity value of the mutation matrix and each mutational signature in a matrix of mutational signatures to form a matrix of similarity values; and (6) selecting one or more mutational signatures from the matrix of mutational signatures when a corresponding cosine similarity value in the matrix of similarity values is greater than or equal to a threshold to indicate a presence of one or more selected mutational signatures in the tumor sample genome. The method may further include normalizing the mutation matrix to form a normalized mutation matrix for the step of determining a cosine similarity value. The normalizing step may further include multiplying the frequency of each trinucleotide by a ratio of a frequency for the trinucleotide in a reference genome to a frequency for the trinucleotide in a portion of the reference genome covered by the targeted panel to form normalized trinucleotide frequencies. The normalizing step may further include scaling the normalized trinucleotide frequencies to values between 0 and 1. The scaling may further include dividing each normalized trinucleotide frequency by a sum of the normalized trinucleotide frequencies. The matrix of mutational signatures may comprise COSMIC mutational signatures from a Catalogue Of Somatic Mutations In Cancer (COSMIC) database. The method may further include determining proportional contributions of the selected mutational signatures by fitting the selected mutational signatures to the normalized mutation matrix. The threshold may have a value of 0.7. The threshold may be a value between 0.6 and 0.99. The method may further include filtering the plurality of variants to form a reduced set of variants for the step of generating a set of trinucleotides.

According to an exemplary embodiment, there is provided a system for analyzing a tumor sample genome for a mutational signature, comprising a processor and a data store communicatively connected with the processor, the processor configured to execute instructions, which, when executed by the processor, cause the system to perform a method, including: (1) amplifying nucleic acid sequences at targeted locations in the tumor sample genome by a targeted panel to generate a plurality of nucleic acid sequence reads; (2) detecting variants in the plurality of nucleic acid sequence reads to produce a plurality of variants: (3) generating a set of trinucleotides by appending a flanking 5′ base and a flanking 3′ base to each variant; (4) determining a frequency of each trinucleotide in the set of trinucleotides to form a mutation matrix; (5) determining a cosine similarity value of the mutation matrix and each mutational signature in a matrix of mutational signatures to form a matrix of similarity values; and (6) selecting one or more mutational signatures from the matrix of mutational signatures when a corresponding cosine similarity value in the matrix of similarity values is greater than or equal to a threshold to indicate a presence of one or more selected mutational signatures in the tumor sample genome. The method may further include normalizing the mutation matrix to form a normalized mutation matrix for the step of determining a cosine similarity value. The normalizing step may further include multiplying the frequency of each trinucleotide by a ratio of a frequency for the trinucleotide in a reference genome to a frequency for the trinucleotide in a portion of the reference genome covered by the targeted panel to form normalized trinucleotide frequencies. The normalizing step may further include scaling the normalized trinucleotide frequencies to values between 0 and 1. The scaling may further include dividing each normalized trinucleotide frequency by a sum of the normalized trinucleotide frequencies. The matrix of mutational signatures may comprise COSMIC mutational signatures from a Catalogue Of Somatic Mutations In Cancer (COSMIC) database. The method may further include determining proportional contributions of the selected mutational signatures by fitting the selected mutational signatures to the normalized mutation matrix. The threshold may have a value of 0.7. The threshold may be a value between 0.6 and 0.99. The method may further include filtering the plurality of variants to form a reduced set of variants for the step of generating a set of trinucleotides.

According to an exemplary embodiment, there is provided a non-transitory machine-readable storage medium comprising instructions which, when executed by a processor, cause the processor to perform a method for analyzing a tumor sample genome for a mutation load, including: (1) receiving a plurality of nucleic acid sequence reads, wherein the nucleic acid sequence reads correspond to a plurality of targeted locations in the tumor sample genome (2) detecting variants in the plurality of nucleic acid sequence reads to produce a plurality of variants; (3) generating a set of trinucleotides by appending a flanking 5′ base and a flanking 3′ base to each variant; (4) determining a frequency of each trinucleotide in the set of trinucleotides to form a mutation matrix; (5) determining a cosine similarity value of the mutation matrix and each mutational signature in a matrix of mutational signatures to form a matrix of similarity values; and (6) selecting one or more mutational signatures from the matrix of mutational signatures when a corresponding cosine similarity value in the matrix of similarity values is greater than or equal to a threshold to indicate a presence of one or more selected mutational signatures in the tumor sample genome. The method may further include normalizing the mutation matrix to form a normalized mutation matrix for the step of determining a cosine similarity value. The normalizing step may further include multiplying the frequency of each trinucleotide by a ratio of a frequency for the trinucleotide in a reference genome to a frequency for the trinucleotide in a portion of the reference genome covered by the targeted panel to form normalized trinucleotide frequencies. The normalizing step may further include scaling the normalized trinucleotide frequencies to values between 0 and 1. The scaling may further include dividing each normalized trinucleotide frequency by a sum of the normalized trinucleotide frequencies. The matrix of mutational signatures may comprise COSMIC mutational signatures from a Catalogue Of Somatic Mutations In Cancer (COSMIC) database. The method may further include determining proportional contributions of the selected mutational signatures by fitting the selected mutational signatures to the normalized mutation matrix. The threshold may have a value of 0.7. The threshold may be a value between 0.6 and 0.99. The method may further include filtering the plurality of variants to form a reduced set of variants for the step of generating a set of trinucleotides.

In various embodiments, nucleic acid sequence data can be generated using various techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, fluorescent-based detection systems, single molecule methods, etc.

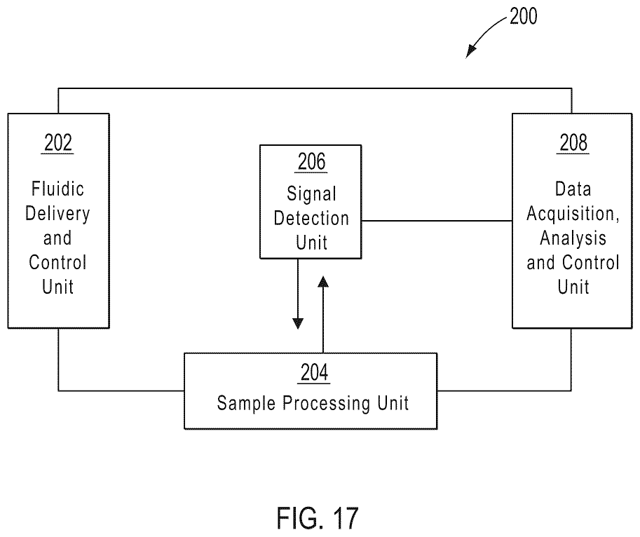

Various embodiments of nucleic acid sequencing platforms, such as a nucleic acid sequencer, can include components as displayed in the block diagram of FIG. 17. According to various embodiments, sequencing instrument 200 can include a fluidic delivery and control unit 202, a sample processing unit 204, a signal detection unit 206, and a data acquisition, analysis and control unit 208. Various embodiments of instrumentation, reagents, libraries and methods used for next generation sequencing are described in U.S. Patent Application Publication No. 2009/0127589 and No. 2009/0026082. Various embodiments of instrument 200 can provide for automated sequencing that can be used to gather sequence information from a plurality of sequences in parallel, such as substantially simultaneously.

In various embodiments, the fluidics delivery and control unit 202 can include reagent delivery system. The reagent delivery system can include a reagent reservoir for the storage of various reagents. The reagents can include RNA-based primers, forward/reverse DNA primers, oligonucleotide mixtures for ligation sequencing, nucleotide mixtures for sequencing-by-synthesis, optional ECC oligonucleotide mixtures, buffers, wash reagents, blocking reagent, stripping reagents, and the like. Additionally, the reagent delivery system can include a pipetting system or a continuous flow system which connects the sample processing unit with the reagent reservoir.

In various embodiments, the sample processing unit 204 can include a sample chamber, such as flow cell, a substrate, a micro-array, a multi-well tray, or the like. The sample processing unit 204 can include multiple lanes, multiple channels, multiple wells, or other means of processing multiple sample sets substantially simultaneously. Additionally, the sample processing unit can include multiple sample chambers to enable processing of multiple runs simultaneously. In particular embodiments, the system can perform signal detection on one sample chamber while substantially simultaneously processing another sample chamber. Additionally, the sample processing unit can include an automation system for moving or manipulating the sample chamber.

In various embodiments, the signal detection unit 206 can include an imaging or detection sensor. For example, the imaging or detection sensor can include a CCD, a CMOS, an ion sensor, such as an ion sensitive layer overlying a CMOS, a current detector, or the like. The signal detection unit 206 can include an excitation system to cause a probe, such as a fluorescent dye, to emit a signal. The expectation system can include an illumination source, such as arc lamp, a laser, a light emitting diode (LED), or the like. In particular embodiments, the signal detection unit 206 can include optics for the transmission of light from an illumination source to the sample or from the sample to the imaging or detection sensor. Alternatively, the signal detection unit 206 may not include an illumination source, such as for example, when a signal is produced spontaneously as a result of a sequencing reaction. For example, a signal can be produced by the interaction of a released moiety, such as a released ion interacting with an ion sensitive layer, or a pyrophosphate reacting with an enzyme or other catalyst to produce a chemiluminescent signal. In another example, changes in an electrical current can be detected as a nucleic acid passes through a nanopore without the need for an illumination source.

In various embodiments, data acquisition analysis and control unit 208 can monitor various system parameters. The system parameters can include temperature of various portions of instrument 200, such as sample processing unit or reagent reservoirs, volumes of various reagents, the status of various system subcomponents, such as a manipulator, a stepper motor, a pump, or the like, or any combination thereof.

It will be appreciated by one skilled in the art that various embodiments of instrument 200 can be used to practice variety of sequencing methods including ligation-based methods, sequencing by synthesis, single molecule methods, nanopore sequencing, and other sequencing techniques.

In various embodiments, the sequencing instrument 200 can determine the sequence of a nucleic acid, such as a polynucleotide or an oligonucleotide. The nucleic acid can include DNA or RNA, and can be single stranded, such as ssDNA and RNA, or double stranded, such as dsDNA or an RNA/cDNA pair. In various embodiments, the nucleic acid can include or be derived from a fragment library, a mate pair library, a ChIP fragment, or the like. In particular embodiments, the sequencing instrument 200 can obtain the sequence information from a single nucleic acid molecule or from a group of substantially identical nucleic acid molecules.

In various embodiments, sequencing instrument 200 can output nucleic acid sequencing read data in a variety of different output data file types/formats, including, but not limited to: *.fasta, *.csfasta, *seq.txt, *qseq.txt, *.fastq, *.sff, *prb.txt, *.sms, *srs and/or *.qv.

FIG. 18 is a schematic diagram of a system for annotating genomic variants, in accordance with various embodiments.

As depicted herein, annotation system 300 can include a nucleic acid sequence analysis device 304 (for example, nucleic acid sequencer, real-time/digital/quantitative PCR instrument, microarray scanner, etc.), an analytics computing server/node/device 302, a display 338 and/or a client device terminal 336, and one or more public 330 and proprietary 332 annotations content sources.

In various embodiments, the analytics computing server/node/device 302 can be communicatively connected to the nucleic acid sequence analysis device 304, client device terminal 336, public annotations content source 330 and/or proprietary annotations content source 332 via a network connection 334 that can be either a “hardwired” physical network connection (for example, Internet, LAN, WAN, VPN, etc.) or a wireless network connection (for example, Wi-Fi, WLAN, etc.).

In various embodiments, the analytics computing device/server/node 302 can be a workstation, mainframe computer, distributed computing node (part of a “cloud computing” or distributed networking system), personal computer, mobile device, etc. In various embodiments, the nucleic acid sequence analysis device 304 can be a nucleic acid sequencer, real-time/digital/quantitative PCR instrument, microarray scanner, etc. It should be understood, however, that the nucleic acid sequence analysis device 304 can essentially be any type of instrument that can generate nucleic acid sequence data from samples obtained from an individual 306.

The analytics computing server/node/device 302 can be configured to host a mapping engine 308. a variant calling engine 310, a decision support module 312 and a reporter module 316.

The mapping engine 308 can be configured to align or map a query nucleic acid sequence read to a reference sequence. Generally, the length of the sequence read is substantially less than the length of the reference sequence. In reference sequence mapping/alignment, sequence reads can be assembled against an existing backbone sequence (for example, reference sequence, etc.) to build a sequence that is similar but not necessarily identical to the backbone sequence. Once a backbone sequence is found for an organism, comparative sequencing or re-sequencing can be used to characterize the genetic diversity within the organism's species or between closely related species. In various embodiments, the reference sequence can be a whole/partial genome, whole/partial exome, whole/partial transcriptome, etc.

In various embodiments, the sequence read and reference sequence can be represented as a sequence of nucleotide base symbols in base space. In various embodiments, the sequence read and reference sequence can be represented as one or more color symbols in color space. In various embodiments, the sequence read and reference sequence can be represented as nucleotide base symbols with signal or numerical quantitation components in flow space.

In various embodiments, the alignment of the sequence read and reference sequence can include a limited number of mismatches between the bases that comprise the sequence read and the bases that comprise the reference sequence. Generally, at least a portion of the sequence read can be aligned to a portion of the reference sequence, such as a reference nuclear genome, a reference mitochondrial genome, a reference prokaryotic genome, a reference chloroplast genome, or the like, in order to minimize the number of mismatches between the sequence fragment and the reference sequence.

The variant calling engine 310 can be configured to receive aligned sequence reads from the mapping engine 308 and analyze the aligned sequence reads to detect and call or identify one or more variants within the reads. Examples of variants that can be called by a variant calling engine 310 include but are not limited to: single nucleotide variants (SNV), single nucleotide polymorphisms (SNP), nucleotide insertions or deletions (indels), copy number variations (CNV) identification, inversion polymorphisms, and the like.

The reporter module 316 can be in communications with the decision support module 312 and be configured to generate a summary report of the called genomic variants that have been annotated by the annotator component 314 that can be part of the decision support module 312.

The decision support module can include an annotator component 314, a variome data store 322, an annotations data store 324, a filtering component 328 and/or an annotations importer component 326. In various embodiments, the annotator component 314 can be in communication with the variant calling engine 310, the variome data store 322 and/or the annotations data store 324. That is, the annotator component 314 can request and receive data and information (through, for example, data streams, data files, text files, etc.) from variant calling engine 310, variome data store 322 and annotations data store 324. In various embodiments, the variant calling engine 310 can be configured to communicate variants called for a sample genome in various formats, such as, but not limited to, variant call format (VCF), generic feature format (GFF) hierarchical data format (HDF), genome variation format (GVF), or HL7 formatted data. It should be understood, however, that the called variants can be communicated using any file format where the called variant information can be parsed and/or extracted for later processing/analysis.

The variome data store 322 can be configured to store the variant calls received from the variant calling engine 310 and/or the annotator component 314 in a format that is accessible for mining.

That is, the called variant data can be maintained as a database or instantiated in some other persistent (and queryable) electronic form in the device memory (for example, hard drive, RAM, ROM, etc.) of the analytics computing server/node/device 302. The called variant data can be structured and use a common syntax and semantic model throughout or include appropriate interpreters between formats that allow for one-to-one mapping between terms and data types. In various embodiments, the variome data store 322 can be an indexed database table of variants. In particular embodiments, the indexed database can be configured for fast querying and filtering operations.

The annotations data store 324 can be in communications with the annotations importer component 326 and be configured to store data and information that can be used by the annotator component 314 to annotate the called variants. That is, the annotations data store 324 can store annotation data and information that can be relevant to the role that the called variant plays in the function, such as at a chromosome level, gene level, a transcript level, a protein level, or the like, (for example, functional type annotations) and/or the biological impact (for example, interpretive type annotations) of the called variants. In various embodiments, functional type annotations can include, but are not limited to: locus classification of the called variant, protein function impact score of the called variant, amino acid changes resulting from the called variant, gene/transcripts affected by the called variant, etc. In various embodiments, interpretive type annotations can include, but are not limited to: disease states or susceptibility to a disease (for example, cancer, diabetes, hypertension, heart disease, etc.) associated with the called variant, impacts that the called variant has on a particular therapeutic regimen (for example, drugs, surgical options, medical device, psychiatric therapy, lifestyle changes, drug sensitivities, etc.), presence of the variant on a list of annotated variants, etc. For example, a SNP variant call can be annotated with functional type annotations that point to the transcripts that the called SNP impacts and interpretive type annotations that are directed to diagnosing a particular disease state or a susceptibility to a disease.

The annotations importer component 326 can be configured to receive annotations content from one or more public 330 or proprietary 332 annotations content sources and convert the annotations content into a format that can be stored in the annotations data store 324 and is accessible for mining. That is, the annotations importer component 326 can convert annotations data and/or information into a format that can be stored onto a database or instantiated in some other persistent (and queryable) electronic form in the device memory (for example, hard drive, RAM, ROM, etc.) of the analytics computing server/node/device 302.

In various embodiments, annotations content can be manually entered or uploaded by a user to the annotations importer component 326 via a computer readable storage medium that is communicatively connected (for example, via a serial data bus connection, parallel data bus connection, internet/intranet network connection, etc.) to the analytics computing server/node/device 302. That is, a user can selectively upload annotations content to the annotations data store 324 depending on the requirements of the particular application. Examples of computer readable medium include, but are not limited to: hard drives, network attached storage (NAS), read-only memory, random-access memory, CD-ROMs, CD-Rs, CD-RWs, magnetic tapes, FLASH memory and other optical/non-optical data storage devices.

In various embodiments, annotations content can be automatically requested and sent from public 330 and/or proprietary 332 annotations content sources to the annotations importer component 326 through the use of a data refresh executable or script. That is, the annotations content in the annotations data store 324 can be continuously refreshed as the public 330 and/or proprietary 332 annotations content sources are updated with new or modified annotations content.

In various embodiments, the annotator component 314 can include a functional annotations engine 318 and interpretive annotations engine 320.

The functional annotations engine 318 can be configured to receive called variants from the variome data store 322, associate one or more functional type annotations (stored in the annotations data store 324) to the called variants and update the called variant records in the variome data store 322 with the associated functional type annotations. In various embodiments, the functional annotations engine 318 can be configured to annotate all called variants that fall within a block of overlapping transcripts (in the sample genome) at the same time. That is, the functional annotations engine 318 can group overlapping transcripts together into a “gene block” and then annotate all variants in the gene block together. The advantage here is that all called variants that are potentially mutually interacting can be grouped and annotated together to give researchers/clinicians greater insight into the synergistic or antagonistic interplay between variants.

In various embodiments, the functional annotations engine 318 can be selectively configured to annotate only called variants that fall within a coding region (for example, exons, codons) of the sample genome being annotated. In various embodiments, the functional annotations engine 318 can be selectively configured to annotate only called variants that fall within an intragenic region, such as an intron, of the sample genome being annotated. In various embodiments, the functional annotations engine 318 can be selectively configured to annotate only the called variants in the intergenic region of the sample genome being annotated.

In various embodiments, the functional annotations engine 318 can receive the called variants in the form of a called variant data file (for example, *.vcf or other file format), associate the functional type annotations, and store the variants and annotations to the variome data store 322. In various embodiments, the functional annotations engine 318 can receive the called variants as variant data (for example, variant base identity and genome position, etc.), associates one or more functional type annotations to the called variant and directly updates the called variant record in the variome data store 322 with the associated functional type annotations information. That is, the functional annotations engine 318 can receive called variants directly from the variome data store 322, annotate them and save them back on the variome data store 322 or alternate data store.

The interpretive annotations engine 320 can be configured to receive called variants from the variome data store 322, associate one or more interpretive type annotations (stored in the annotations data store 324) to the called variants and update the called variant records in the variome data store 322 with the associated interpretive type annotations.

In various embodiments, the interpretive annotations engine 320 receives the called variants in the form of a called variant data file (for example, *.vcf or other file format), associate the interpretive type annotations, and store the variants and annotations to the variome data store 322. In various embodiments, the interpretive annotations engine 318 receives the called variants as variant data (for example, variant base identity and genome position, etc.), associates one or more interpretive type annotations to the called variant and directly updates the called variant record in the variome data store 322 with the associated interpretive type annotations information.

In various embodiments, the system can be configured to automate the processing of sample data. For example, a workflow can be selected to define how the data is processed by the mapping engine 308, the variant calling engine 310, and the annotator component 314. In particular embodiments, a workflow can be selected when setting up the run on the nucleic acid sequence analysis device 304 and the data can be automatically uploaded to the analytics computing device 302. Additionally, the workflow can be automatically launched when the data has been uploaded. In other embodiments, the data can be uploaded, manually or automatically, from the nucleic acid sequence analysis device 304 and the workflow can be selected and launched manually. Generally, once the workflow has been selected and launched, analysis can proceed from through the mapping engine 308, the variant calling engine, 310, and the annotator component 314 without further intervention by a user.

The filtering component 328 can be configured to allow a user to set filter conditions to filter the called variants that are included in the summary report generated by the reporter module 316. Examples of filter conditions include, but are not limited to, filtering for: variants that are nonsynonymous and fall within a particular gene, variants that are associated with a particular disease condition, variants that have a functional score of greater or less than a selected value, novel variants that are not present in a functional type annotations source, variants that fall in gene panel regions (defined by user), etc. In various embodiments, the filtering component 328 can utilize combinations of filters, such as for example filtering for variants that fall within a particular gene and have a functional score indicative of a significant effect.

In various embodiments, the filtering component 328 can be configured with a collection of filters to select for variants with a high likelihood of having possible functional significance. For example, the filtering component 328 can select for missense mutations and nonsense mutations and exclude synonymous mutations. Still further, the filtering component 328 can select for variants that affect allele frequency. Also, the filtering component 328 may select or exclude variants at positions of known significance, such as positions known to have a high incidence of mutation in cancers, positions with a low or high number of false positive variant calls, positions known to have a minimal functional impact, or the like.

In various embodiments, the variome data 322 and the annotations data 324 stores can be combined into a single data store configured to store both called variant data and variant annotations information.

Client terminal 336 can be a thin client or thick client computing device. In various embodiments, client terminal 336 can have a web browser (for example, INTERNET EXPLORER™ FIREFOX™, SAFARI™, etc.) that can be used to communicate information to and/or control the operation of the mapping engine 308, variant calling engine 310, decision support module 312, annotator component 314, filtering component 328, annotations importer component 326, variome data store 322, annotations data store 324, functional annotations engine 318 and/or interpretive annotations engine 320 using a browser to control their function. For example, the client terminal 336 can be used to configure the operating parameters (for example, match scoring parameters, annotations parameters, filtering parameters, data security and retention parameters, etc.) of the various modules, depending on the requirements of the particular application. Similarly, client terminal 336 can also be configured to display the results of the analysis performed by the decision support module 312 and the nucleic acid sequencer 304.

It should be understood that the various data stores disclosed as part of system 300 can represent hardware-based storage devices (for example, hard drive, flash memory, RAM, ROM, network attached storage, etc.) or instantiations of a database stored on a standalone or networked computing device(s).

It should also be appreciated that the various data stores and modules/engines shown as being part of the system 300 can be combined or collapsed into a single module/engine/data store, depending on the requirements of the particular application or system architecture. Moreover, in various embodiments, the system 300 can comprise additional modules, engines, components or data stores as needed by the particular application or system architecture or to extend functionality.

In various embodiments, the system 300 can be configured to process the nucleic acid reads in color space. In various embodiments, system 300 can be configured to process the nucleic acid reads in base space. In various embodiments, system 300 can be configured to process the nucleic acid sequence reads in flow space. It should be understood, however, that the system 300 disclosed herein can process or analyze nucleic acid sequence data in any schema or format as long as the schema or format can convey the base identity and position (or position range) of the nucleic acid sequence within the reference sequence.

In various embodiments, the system 300 can be configured to distinguish between positions with a called variant, positions that have been called as reference, and positions with no call. Positions with a called variant can include positions where sufficient evidence was provided by the reads to indicate the specimen sequence contains a variant. Positions that have been called as reference can include positions where there is sufficient evidence to support the conclusion that the specimen sequence is substantially identical to the reference sequence at the position. Positions with no call can include positions where there is insufficient evidence to determine if the specimen sequence is the same as or different from the reference sequence. For example, positions with no call can include positions with low coverage, positions with low base quality, or positions where the read sequences indicate different bases with insufficient homogeneity to determine the sequence with sufficient confidence. Generally, positions with no call can be indicated as matching the reference sequence and may be excluded from reporting of variants.

According to various exemplary embodiments, one or more features of any one or more of the above-discussed teachings and/or exemplary embodiments may be performed or implemented using appropriately configured and/or programmed hardware and/or software elements. Determining whether an embodiment is implemented using hardware and/or software elements may be based on any number of factors, such as desired computational rate, power levels, heat tolerances, processing cycle budget, input data rates, output data rates, memory resources, data bus speeds, etc., and other design or performance constraints.

Examples of hardware elements may include processors, microprocessors, input(s) and/or output(s) (I/O) device(s) (or peripherals) that are communicatively coupled via a local interface circuit, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. The local interface may include, for example, one or more buses or other wired or wireless connections, controllers, buffers (caches), drivers, repeaters and receivers, etc., to allow appropriate communications between hardware components. A processor is a hardware device for executing software, particularly software stored in memory. The processor can be any custom made or commercially available processor, a central processing unit (CPU), an auxiliary processor among several processors associated with the computer, a semiconductor based microprocessor (e.g., in the form of a microchip or chip set), a macroprocessor, or generally any device for executing software instructions. A processor can also represent a distributed processing architecture. The I/O devices can include input devices, for example, a keyboard, a mouse, a scanner, a microphone, a touch screen, an interface for various medical devices and/or laboratory instruments, a bar code reader, a stylus, a laser reader, a radio-frequency device reader, etc. Furthermore, the I/O devices also can include output devices, for example, a printer, a bar code printer, a display, etc. Finally, the I/O devices further can include devices that communicate as both inputs and outputs, for example, a modulator/demodulator (modem; for accessing another device, system, or network), a radio frequency (RF) or other transceiver, a telephonic interface, a bridge, a router, etc.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. A software in memory may include one or more separate programs, which may include ordered listings of executable instructions for implementing logical functions. The software in memory may include a system for identifying data streams in accordance with the present teachings and any suitable custom made or commercially available operating system (O/S), which may control the execution of other computer programs such as the system, and provides scheduling, input-output control, file and data management, memory management, communication control, etc.

According to various exemplary embodiments, one or more features of any one or more of the above-discussed teachings and/or exemplary embodiments may be performed or implemented using appropriately configured and/or programmed non-transitory machine-readable medium or article that may store an instruction or a set of instructions that, if executed by a machine, may cause the machine to perform a method and/or operations in accordance with the exemplary embodiments. Such a machine may include, for example, any suitable processing platform, computing platform, computing device, processing device, computing system, processing system, computer, processor, scientific or laboratory instrument, etc., and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, floppy disk, read-only memory compact disc (CD-ROM), recordable compact disc (CD-R), rewriteable compact disc (CD-RW), optical disk, magnetic media, magneto-optical media, removable memory cards or disks, various types of Digital Versatile Disc (DVD), a tape, a cassette, etc., including any medium suitable for use in a computer. Memory can include any one or a combination of volatile memory elements (e.g., random access memory (RAM, such as DRAM, SRAM, SDRAM, etc.)) and nonvolatile memory elements (e.g., ROM, EPROM, EEROM, Flash memory, hard drive, tape, CDROM, etc.). Moreover, memory can incorporate electronic, magnetic, optical, and/or other types of storage media. Memory can have a distributed architecture where various components are situated remote from one another, but are still accessed by the processor. The instructions may include any suitable type of code, such as source code, compiled code, interpreted code, executable code, static code, dynamic code, encrypted code, etc., implemented using any suitable high-level, low-level, object-oriented, visual, compiled and/or interpreted programming language.

According to various exemplary embodiments, one or more features of any one or more of the above-discussed teachings and/or exemplary embodiments may be performed or implemented at least partly using a distributed, clustered, remote, or cloud computing resource.

According to various exemplary embodiments, one or more features of any one or more of the above-discussed teachings and/or exemplary embodiments may be performed or implemented using a source program, executable program (object code), script, or any other entity comprising a set of instructions to be performed. When a source program, the program can be translated via a compiler, assembler, interpreter, etc., which may or may not be included within the memory, so as to operate properly in connection with the O/S. The instructions may be written using (a) an object oriented programming language, which has classes of data and methods, or (b) a procedural programming language, which has routines, subroutines, and/or functions, which may include, for example, C, C++, R, Pascal, Basic, Fortran, Cobol, Perl, Java, and Ada.

According to various exemplary embodiments, one or more of the above-discussed exemplary embodiments may include transmitting, displaying, storing, printing or outputting to a user interface device, a computer readable storage medium, a local computer system or a remote computer system, information related to any information, signal, data, and/or intermediate or final results that may have been generated, accessed, or used by such exemplary embodiments. Such transmitted, displayed, stored, printed or outputted information can take the form of searchable and/or filterable lists of runs and reports, pictures, tables, charts, graphs, spreadsheets, correlations, sequences, and combinations thereof, for example.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for analyzing a tumor sample genome for a mutational signature, comprising:
amplifying nucleic acid sequences at targeted locations in the tumor sample genome by a targeted panel to generate a plurality of amplified target sequences, wherein the tumor sample has a lower sample input than a sample input required for whole exome sequencing or whole genome sequencing:
sequencing the amplified target sequences to generate a plurality of nucleic acid sequence reads;
detecting variants in the plurality of nucleic acid sequence reads to produce a plurality of variants:
generating a set of trinucleotides by appending a flanking 5′ base and a flanking 3′ base to each variant:
determining a frequency of each trinucleotide in the set of trinucleotides to form a mutation matrix:
determining a cosine similarity value of the mutation matrix and each mutational signature in a matrix of mutational signatures to form a matrix of similarity values; and
selecting one or more mutational signatures from the matrix of mutational signatures when a corresponding cosine similarity value in the matrix of similarity values is greater than or equal to a threshold to indicate a presence of one or more selected mutational signatures in the tumor sample genome.

2. The method of claim 1, further comprising normalizing the mutation matrix to form a normalized mutation matrix for the step of determining a cosine similarity value.

3. The method of claim 2, wherein the normalizing step further comprises multiplying the frequency of each trinucleotide by a ratio of a frequency for the trinucleotide in a reference genome to a frequency for the trinucleotide in a portion of the reference genome covered by the targeted panel to form normalized trinucleotide frequencies.

4. The method of claim 3, wherein the normalizing step further comprises scaling the normalized trinucleotide frequencies to values between 0 and 1.

5. The method of claim 4, wherein the scaling further comprises dividing each normalized trinucleotide frequency by a sum of the normalized trinucleotide frequencies.

6. The method of claim 1, wherein the matrix of mutational signatures comprises COSMIC mutational signatures from a Catalogue Of Somatic Mutations In Cancer (COSMIC) database.

7. The method of claim 2, further comprising determining proportional contributions of the selected mutational signatures by fitting the selected mutational signatures to the normalized mutation matrix.

8. The method of claim 1, wherein the threshold is 0.7.

9. The method of claim 1, wherein the threshold is between 0.6 and 0.99.

10. The method of claim 1, further comprising filtering the plurality of variants to form a reduced set of variants for the step of generating a set of trinucleotides.

11. A system for analyzing a tumor sample genome for a mutational signature, comprising a processor and a data store communicatively connected with the processor, the processor configured to execute instructions, which, when executed by the processor, cause the system to perform a method, including:
amplifying nucleic acid sequences at targeted locations in the tumor sample genome by a targeted panel to generate a plurality of amplified target sequences, wherein the tumor sample has a lower sample input than a sample input required for whole exome sequencing or whole genome sequencing:
sequencing the amplified target sequences to generate a plurality of nucleic acid sequence reads;
detecting variants in the plurality of nucleic acid sequence reads to produce a plurality of variants:
generating a set of trinucleotides by appending a flanking 5′ base and a flanking 3′ base to each variant:
determining a frequency of each trinucleotide in the set of trinucleotides to form a mutation matrix:
determining a cosine similarity value of the mutation matrix and each mutational signature in a matrix of mutational signatures to form a matrix of similarity values: and
selecting one or more mutational signatures from the matrix of mutational signatures when a corresponding cosine similarity value in the matrix of similarity values is greater than or equal to a threshold to indicate a presence of one or more selected mutational signatures in the tumor sample genome.

12. The system of claim 11, further comprising normalizing the mutation matrix to form a normalized mutation matrix for the step of determining a cosine similarity value.

13. The system of claim 12, wherein the normalizing step further comprises multiplying the frequency of each trinucleotide by a ratio of a frequency for the trinucleotide in a reference genome to a frequency for the trinucleotide in a portion of the reference genome covered by the targeted panel to form normalized trinucleotide frequencies.

14. The system of claim 13, wherein the normalizing step further comprises scaling the normalized trinucleotide frequencies to values between 0 and 1.

15. The system of claim 14, wherein the scaling further comprises dividing each normalized trinucleotide frequency by a sum of the normalized trinucleotide frequencies.

16. The system of claim 11, wherein the matrix of mutational signatures comprises COSMIC mutational signatures from a Catalogue Of Somatic Mutations In Cancer (COSMIC) database.

17. The system of claim 12, further comprising determining proportional contributions of the selected mutational signatures by fitting the selected mutational signatures to the normalized mutation matrix.

18. The system of claim 11, wherein the threshold is 0.7.

19. The system of claim 11, wherein the threshold is between 0.6 and 0.99.

20. The system of claim 11, further comprising filtering the plurality of variants to form a reduced set of variants for the step of generating a set of trinucleotides.
